(19) United States
(12) Patent Application Publication
(10) Pub. No.: US 2021/0128786 A1
(43) Pub. Date: May 6, 2021

(54) AVIAN BASED LUNG ASSIST DEVICE

(71) Applicant: University of Vermont and State Agricultural College, Burlington, VT (US)

(72) Inventors: Daniel J. WEISS, Essex Junction, VT (US); Sean WRENN, South Burlington, VT (US); Franziska Elisabeth UHL, Burlington, VT (US); Ethan GRISWOLD, Waterbury Center, VT (US); Patrick LEE, South Burlington, VT (US); Darcy WAGNER, Munich (DE); Dryver Roy HUSTON, South Burlington, VT (US)

(21) Appl. No.: 16/482,636

(22) PCT Filed: Jan. 30, 2018
(86) PCT No.: PCT/US18/15979

§ 371 Date: Jul. 31, 2019

Related U.S. Application Data
(60) Provisional application No. 62/452,685, filed on Jan. 31, 2017.

(51) Int. Cl. A61L 27/38; A61L 27/36; C12N 5/071; A61M 1/16; A61M 16/08

(52) U.S. Cl. CPC A61L 27/3886; A61L 27/3687; C12N 5/0688; A61M 1/1698; A61M 16/208; A61M 16/0883; C12N 2533/90; C12N 2513/00; A61M 2210/1039; A61M 1/1678

(57) ABSTRACT

Compositions, materials, devices and methods are disclosed for the use of decellularized avian lung scaffolds for potential xenotransplantation and other uses including use as novel lung assist or bridge-to-transplant devices and as potential alternatives to current ECMO devices and technologies. Decellularization of an avian lung and recellularization with human lung cells is described.

### CROSS-REFERENCE TO RELATED APPLICATIONS

This application claims priority to U.S. Provisional application No. 62/452,685, filed on Jan. 31, 2017, the disclosure of which is incorporated herein by reference.

### STATEMENT REGARDING FEDERALLY SPONSORED RESEARCH

This invention was made with government support under grant number R21-EB024329 awarded by the National Institutes of Health. The government has certain rights in the invention.

### BACKGROUND OF THE INVENTION

Allogeneic lung transplant remains the final available treatment modality and potentially life-saving intervention for patients with end stage lung diseases. However lung transplantation remains limited by a shortage of suitable donor lungs and many patients with end-stage lung diseases will succumb while on transplant waiting lists. There are few available bridging devices, comparable to left ventricular assist devices used in end stage cardiac disease patients, for use in end stage lung disease patients. Extracorporeal membrane oxygenation (ECMO) devices have a significant role in short term acute neonatal respiratory diseases and a more limited role in acute adult respiratory diseases. However, ECMO devices require hospitalization in critical care units and specialized health care providers. It is not a practical or cost effective option for long term bridging to lung transplant or as long term support for end stage lung disease patients who do not qualify for transplantation. As such, there is a critical need for practical, easy to use devices.

### SUMMARY OF THE DISCLOSURE

In the present disclosure, we provide compositions, materials, devices and methods for the use of decellularized avian lung scaffolds for potential xenotransplantation and other uses including use as novel lung assist or bridge-to-transplant devices and as potential alternatives to current ECMO devices and technologies. For example, the decellularized avian lungs can be recellularized with human lung cells including but not limited to differentiated airway and/or alveolar and pulmonary vascular endothelial cells and lung stem and progenitor cells. The cells may be derived from embryonic or induced pluripotent stem cells differentiated into functional lung cells. We have successfully decellularized and characterized both small and large avian lungs and demonstrate recellularization with a range of human lung cell types.

Representative small (chicken) and large (emu) avian lungs were decellularized utilizing detergent-based protocols. Light and electron microscopy, quantitation and gel analyses of residual DNA, and immunohistochemical (IHC) and mass spectrometric analyses of remaining extracellular matrix (ECM) and other proteins demonstrated maintenance of lung structure, minimal residual DNA, and retention of major ECM proteins in the decellularized scaffolds. Seeding with human bronchial epithelial cells, human pulmonary vascular endothelial cells, human mesenchymal stromal cells, and human lung fibroblasts demonstrated robust recellularization and growth. These studies demonstrate that decellularized avian lungs can be used for generating functional lung tissue for clinical therapeutics.

### BRIEF DESCRIPTION OF THE FIGURES

FIG. 1. Chicken and emu bird lungs are comparably grossly decellularized and silicone injection molds were created to analyze chicken anatomy. A) Progressive decellularization results in comparable clearing of blood and pink coloration resulting in final pearly white translucent tissues. Representative images from chicken (n=14) and emu (n=7) bird lungs are shown. SDC=sodium deoxycholate, NaCl=sodium chloride, DNase=DNase solution, PAA=peracetic acid; B) Two silicone injection molds of chicken airways and airs sacs were cast. The first mold with air sacs attached is shown dorsally (I) and ventrally (II). The second mold without air sacs is shown from the same views (III and IV respectively), with a highly magnified ventral view of the parabronchi and parabronchia gas exchange microstructures (atria) (V).

FIG. 2. The decellularization process largely preserves the native structure of bird lungs. Representative images of native and decellularized chicken (A) and emu (B) bird lungs are depicted. Photomicrographs demonstrate qualitative preservation of characteristic structure and major ECM proteins (collagen, elastin) by H&E, EVG, and trichrome stains. Glycosaminoglycan content is qualitatively decreased as assessed by Alcian blue staining. a=airways, bv=blood vessels. Representative images from chicken (n=14) and emu (n=4) bird lungs are shown. Original magnification 40× and 400×, scale bar is indicated on each image.

FIG. 3. The decellularization process largely preserves the ultrastructure of bird lung extracellular matrix. Transmission electron microscopy demonstrates comparable appearance of the parabronchial microstructures in decellularized chicken (A) and emu (B) bird lungs. Representative images from a single decellularized chicken and single emu bird lung are shown. Enlargements of the inserts for each image demonstrate more structural details. Collagen fibers are indicated by arrows and capillaries are indicated by “c”. Original magnification and scale bar is indicated on each image).

FIG. 4. DNA gels demonstrate minimal residual DNA in decellularized chicken and emu bird lungs compared to native controls. A DNA ladder (M) and salmon sperm DNA (ssD, positive control) are shown for comparison. Nat=native, Representative gel and also quantitation of the DNA content in respective representative native and decellularized chicken (6) and emu (4) bird lungs are shown.

FIG. 5. Decellularization preserves major ECM proteins in chicken and emu bird lungs. Representative photomicrographs comparing native and decellularized chicken (A) and emu (B) bird lungs are depicted and demonstrate similar qualitative retention of major structural ECM proteins. Nuclear DAPI staining is depicted in blue and the stain of interest is depicted in red. Col1=type I collagen, Col4=type 4 collagen, Elast=elastin, Fib=fibronectin, Lam=laminin, bv=blood vessel, a=airway. Original magnifications 200×, scale bar 200 μm. a=airways, bv=blood vessels. Representative images from 2 chicken and 2 emu bird lungs are shown.

FIG. 6. Mass spectrometric assessment of residual proteins following decellularization of chicken and emu bird lungs demonstrates overall concordance in residual proteins detected. Positively identified proteins in decellularized chicken (A) and emu (B) bird lungs (i.e. those proteins which were detected with at least 2 unique peptide hits and exceeded the FDR cutoff for identification) were assigned to groups according to subcellular location (cytoskeletal, cytosolic, ECM, membrane-associated, nuclear, secreted, and uncharacterized in case no subcellular location was specified). Heatmaps were generated using the log 2 transformation of total peptide counts for all positively identified proteins and grouped by category. Representative heatmaps from 6 chicken and 4 emu bird lungs are shown. Of note, only a limited database for emu proteins is available for reference.

FIG. 7. HBEs, hMSCs, CBFs, and HLFs demonstrate comparable initial seeding patterns, different growth patterns following inoculation into decellularized chicken and emu lungs. Representative H&E low power (100×) photomicrographs show characteristic recellularization patterns one day post-inoculation of each cell type (left column), day 7 (middle column), and the last day viable cells were observed (right column) in acellular chicken (A) or emu (B) bird lungs. Representative images from 3-4 chicken lungs and 3-6 emu bird lung segments seeded with each cell type are shown. In general, cells that do not interact with the ECM scaffold and remain in the airspaces or vascular spaces unattached to any matrix demonstrated rounding up of cells and nuclear fragmentation, consistent with anoikis or apoptosis. Arrowheads indicate fragmented nuclei in cells that appear to be undergoing apoptosis. Arrows indicate cells that have no clear blue nuclear staining and thus appear not to be alive. Stars indicate locations of rounded and detached cells. Original magnification 200×, scale bars are indicated on each image.

FIG. 8. Cells seeded into decellularized chicken or emu lungs demonstrate similar patterns of Ki67 and caspase-3 staining. Representative photomicrographs of Ki67 (A) and caspase-3 (B) staining day 1 and day 7 post-inoculation of each cell type. Ki67 or caspase-3 staining is indicated in red and DAPI nuclear staining in blue. Representative images from 3 decellularized chicken and emu lungs seeded with each cell type are depicted. Original magnification 200×, scale bar 200 μm. (C) Quantitative analysis of randomized images from 2 decellularized chicken lungs and 1 emu lung segment seeded with each individual cell type. 4 regions/slide from each seeding and time point were quantified to determine the percentage of ratio of positive stained Ki67 or caspase-3 expressing cells (red staining=Ki67/caspase-3) to total cells (blue staining=DAPI). Mean±standard deviations of the different quantified regions are depicted.

FIG. 9. Functional diagram of a device according to an embodiment of the present disclosure.

FIG. 10. Functional diagram of a device according to another embodiment of the present disclosure.

FIG. 11. Functional diagram of a portable device according to another embodiment of the present disclosure.

FIG. 12. Controls for immunohistological staining. A) No primary antibody control on native chicken and emu lung tissue for the respective antibodies indicated above each image. B) Antibody positive controls with and without primary antibody using human gall bladder, kidney, liver, small bowel, and tonsil tissue. Collagen I, IV, laminin, fibronectin, elastin, Ki67, and caspase 3=red, DAPI=blue. Original magnification: 200×, scale bar: 100 μm.

FIG. 13. Minimal residual anionic detergent (SDC) is detected in effluents from either chicken (A) or emu (B) lungs at the conclusion of the decellularization protocol. SDC concentration was calculated using a SDC standard curve (n=7 for chicken and n=1 for emu).

FIG. 14. Table showing total peptide counts for positively identified proteins in individual chicken bird lung samples, C=chicken.

FIG. 15. Table showing total peptide counts for positively identified proteins in individual emu bird lung samples.

FIG. 16. Cannula positioning for avian lung decellularization. Connectors were placed in the main bronchi (B), pulmonary artery (PA) and pulmonary vein (PV)

FIG. 17. Step-by-step decellularization process. Representative images of the evolution and sequential perfusion of reagents in chicken lungs.

FIG. 18. Chicken lung histology and remaining DNA. Acellular sections of chicken lungs before (native) and after the decellularization process (a). Acellular chicken lungs showed less than 50 ng/mg of remaining DNA (b).

FIG. 19. Endothelial cells after 3 days. Representative images of cell attachment in chicken lungs recellularized with HUVEC, CBF and hMSC cells.

FIG. 20. Epithelial cells after 3 days. Representative images of cell attachment in chicken lungs recellularized with HBE and HLF cells.

### DETAILED DESCRIPTION OF THE INVENTION

The present disclosure provides decellularized avian lungs, recellularized with mammalian lung cells, such as human lung cells. The decellularized and recellularized avian lungs can be used as gas exchange units for use in pulmonary therapeutics. The present disclosure also provides devices incorporating the decellularized, recellularized avian lungs. The devices can be used as extracorporeal lung assist devices or can be implanted to augment the function of a host's lung.

To prepare an avian lung for incorporation into a device, an avian lung lacking cells (decellularized) but in which the scaffolding is maintained, is first prepared. To prepare decellularized avian lung scaffolding, any bird can be used. The size of the bird is not limiting and both small and large bird lungs can be used. Examples of suitable bird lungs include: chicken, turkey, emu, and ostrich, among others. The size of the bird lung to be utilized may depend on the intended use—for example large lungs can be used for ECMO-type devices to be utilized in intensive care unit settings; small-medium sized lungs can be used for portable use, small lungs can be used for potential implantable use. A bird can be euthanized and its lung identified. A lung/trachea/heart block may be obtained or just the lung may be isolated and removed. Vessels and/or ducts of the lung can be cannulated using methods and materials known in the art. Cannulated lungs can be flushed with suitable sterile solution to clear the blood. For example, the lung may be cannulated and flushed with wash solutions, such as sterile normal saline (or a buffer) with or without an anti-coagulant (such as heparin). Following cannulation and washing, the lung can be perfused via the cannula with a cell disruption medium for effecting decellularization.

The cell disruption medium for decellularization can comprise one or more detergents. The lung tissue can be exposed to the medium via perfusion. Perfusion can be carried out with more than one type of cell disruption medium used sequentially. Perfusion through the tissue can be carried out in any direction. For example, perfusion can be antegrade or retrograde, and directionality can be alternated if desired. Perfusion with each perfusion solution can be carried out for 2 to 48 hours, but generally ranges from 2 to 24 hours. The entire treatment time including washes etc. can be up to 72 hours (e.g., 2 to 72 hours).

The detergents are generally used below their critical micelle concentration (CMC), although they can be used at or above CMC also. Detergents can be denaturing or non-denaturing (with respect to proteins). Denaturing detergents can be anionic such as sodium dodecyl sulfate (SDS), or cationic such as ethyl trimethyl ammonium bromide. Non-denaturing detergents can be non-ionic such as Triton X or Tween, or zwitterionic such as 3-[(3-cholamidopropyl)dimethylammonio]-1-propanesulfonate (CHAPS). Examples of suitable detergents for cell disruption include, but are not limited to, Triton X-100, Triton X-114, NP-40, Brij-35, Brij-58, Tween 20, Tween 80, Octyl glucoside, SDS, CHAPS, 3-[(3-cholamidopropyl)dimethylammonio]-2-hydroxy-1-propanesulfonate (CHAPSO), and polyethylene glycol (PEG). These detergents are commercially available. Exemplary methods for use of detergents for decellularization of organs and tissues are disclosed in U.S. Pat. Nos. 8,470,520, and 9,005,885, the descriptions of which methods are incorporated herein by reference. Examples of detergent concentrations useful for lung decellularization include Examples of detergent concentrations useful for lung decellularization include 0.01 to 2% sodium deoxycholate (SDC), 0.01 to 1% sodium dodecyl sulfate (SDS), and 0.01 to 1% Triton X-100 (and all concentrations to the hundredth decimal point therebetween).

Decellularization can also be effected by subjecting the tissue to repeated freeze-thaw cycles (such as by using cooling agents such as liquid nitrogen) or by using a medium which has water or buffer at an osmolarity that is incompatible with cells. The tissue can also or alternatively be treated with nuclease (e.g., ribonuclease, deoxyribonuclease), protease, collagenase or combinations of these enzymes.

Prior to treatment with detergents, between different treatments, and after the treatments, the lung can be washed in sterile water or buffer (such as phosphate buffered saline (PBS)) with mild agitation. Washing can additionally or alternatively be carried out by using hypertonic or salt solution. Wash steps can be done with solutions that include antibiotics, such as penicillin, streptomycin, gentamicin, amphotericin B and the like. Decellularization of the lungs can be assessed by any method directed to assessing integrity of cells or the presence of nuclei. For example, decellularization and the integrity of the remaining scaffolding can be assessed by one or more of: a) histology (light and electron microscopy); b) histochemistry, immunohistochemistry and/or western blotting for major remaining ECM proteins and glycoproteins; c) mass spectrometry for full assessment of remaining proteins and glycoproteins; d) DNA quantitation and gel analyses; e) quantification of residual detergents. For example, one or more in a panel of commercially available antibodies and other reagents capable of detecting relevant avian ECM and other proteins (R and D Systems, Minneapolis, Minn.) can be used. There is significant homology between the mammalian and avian ECM proteins. Commercially available antibodies that react with bird proteins include but are not limited to: purified mouse anti-fibronectin monoclonal (610077—1:100—BD Transduction Laboratories, Franklin Lakes, N.J., USA), laminin antibody polyclonal (ab11575—1:100—Abcam, Cambridge, United Kingdom), rabbit polyclonal to alpha elastin (ab21607—1:100—Abcam), smooth muscle myosin heavy chain 2 polyclonal (ab53219—1:100—Abcam), collagen I polyclonal (ab292—1:100—Abcam), Ki67 proliferation marker polyclonal (ab16667—1:50—Abcam), cleaved caspase-3 polyclonal (Asp175—1:100—Cell Signaling Technology, Danvers, Mass., USA), mouse clone anti-human actin polyclonal (1A4—1:10,000—Dako via FAHC, Denmark).

Examples of criteria that can be used to confirm decellularization include absence of detectable nuclei (such as by hematoxylin/eosin staining or by high resolution microscopy such as electron microscopy), lack of detectable DNA (such as lack of visible DNA on a DNA gel), or confirming that the amount of measured DNA on a DNA gel is less than 100 or 50 ng/mg of tissue (Crapo et al., 2011, 32:3233-43). One or more of the above criteria may be used. Any other criteria indicating the lack of viable intact cells can also be used.

Decellularized lungs can be used immediately for recellularization or can be stored until further use. For example, the decellularized lung can be flushed with PBS storage solution: 1× Phosphate Buffered Saline Solution (Corning, Corning, N.Y., USA) supplemented with Penicillin/Streptomycin (500 IU/mL Penicillin/500 μg/mL Streptomycin, Lonza, Basel, Switzerland), Gentamicin (50 mg/L, Corning), and Amphotericin B (2.5 mg/L, Corning) and stored in storage solution at 4° C. until further processing or usage for reseeding. Lungs can be stored for at least up to 3 months.

Prior to recellularization, the lungs can be coated with an alginate or other materials to produce an air and liquid-tight external seal. For example, the lungs can be coated with sodium alginate (such as with a 1 to 5% solution of sodium alginate, Manugel, FMC Biopolymer, Philadelphia, Pa., USA) which is then cross-linked (such as with a calcium chloride solution (such as 1 to 5% solution). This results in segments being uniformly coated in a calcium alginate hydrogel that serves as an artificial pleural sealant. Other materials that can be used for external coating include but are not limited to commercially available alginate, cellulose, chitosan, gelatin, hyaluronic acid, and starch, (i.e. medical grade) of various molecular weights either functionalized by methacrylation, oxidation, or other chemical reaction or by conjugation or other chemical reaction with amino acids, catechols, and other molecules that can impart adhesive and rapid gelling properties

For recellularization, cell suspensions comprising desired cells in suitable media can be delivered to the decellularized lungs. Suitable media include normal saline, PBS or any other cell culture media.

The cells used for recellularization can be differentiated or regenerative. For example, cells may be progenitor cells, precursor cells, stem cells (adult or fetal), differentiated or committed cell types. Stem cells can be human induced pluripotent stem cells (iPSC), mesenchymal stem cells, human umbilical vein endothelial cells, multipotent adult progenitor cells (MAPC), or embryonic stem cells. Regenerative cells may be derived from lungs or other tissues. For example, cell suspensions for recellularization can comprise, or consist essentially of (meaning those are the only cell types present in the suspension): human bronchial epithelial (HBE) cells; human pulmonary vascular endothelial cells; human lung fibroblasts (HLF); human bone marrow-derived mesenchymal stromal cells (hMSCs), pulmonary endothelial colony forming cells (CBF) and the like. The cells may be freshly isolated cells, primary cells, secondary cells or may be obtained from cell lines. The cells can be proliferated in vitro prior to use for recellularization. Cells may be added either as individual cell types or in different combinations.

The desired number of cells can be used for recellularization (which may also be referred to herein as seeding). For example, 1×10⁴ to 1×10¹⁰ cells in a suitable amount of media (such as 0.1 to 2.0 ml) can be administered per lung by airway (e.g., HBE, HLF, hMSC) or vascular (e.g., CBF) inoculation into the alginate-coated lungs. For example, 4.5-5.0×10⁷ cells per lung, suspended in 1.0 ml media of each cell type can be administered. Different numbers of cells, different amounts of media, or different routes of administration for the different cell types can also be utilized. Lungs can be incubated in relevant basal media under static conditions at 37° C. overnight (or from 2 to 24 hours) to allow initial cell adherence.

Recellularization can be assessed as follows. Seeded lungs can be assessed by light microscopy (H and E staining of paraformaldehyde-fixed, paraffin-embedded sections) of sample tissue from the lung or from lungs processed in parallel. Numbers of individual cell types and qualitative determinations of recellularization patterns can be systematically and comparatively assessed on serial sections from each slice. Assessments of proliferation and of early apoptosis by immunohistochemical staining (IHC) for Ki-67 and caspase-3, respectively, can be utilized to assess cell fates over time. Quantitative PCR (qPCR) and cell-specific IHC, can be utilized to assess the seeded cells. These assessments are done by using standard techniques known in the art. Recellularization can be studied on an ongoing basis and recellularized lungs can be used for clinical use once near complete (approximately 90% or more) or complete coverage of the decellularized scaffold surface area has been achieved.

Recellularized lungs can be used immediately for encasing in a housing for use as a lung assist device or can be stored until further use. The housing may be made by using techniques such as 3D bio-printing, injection molding, or compression molding. Suitable materials for housing include biocompatible polymers such as polylactide (PLA), polyglycolide (PGA), polycaprolactone (PCL), and their copolymers. Other suitable materials are known in the art. (e.g., See Shastri et al., Current Pharmaceutical Biotechnology, 2003, 4:331-337; Chen et al., Progress in Polymer Science, 2008, 33:1059-1087). For implantable versions, multi-phase structures can be prepared to satisfy various functional requirements: biocompatibility, durability, and flexibility by blending or multi-layering various biopolymers. Blood and gas leak tests can be performed with respect to various flow rates and operating flow rate ranges of blood and air for the lung without bursting can be determined. Examples of blood flow or fluid flow rates include 1-10 liters/minute and examples of gas flow rates include 1-15 liters/minute.

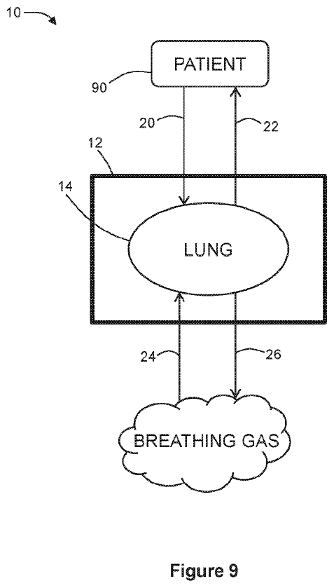

With reference to FIG. 9, the present disclosure may be embodied as a device 10 for lung supplementation or replacement. The device 10 includes an isolated avian lung 14 and a housing 12 (also referred to herein as a vessel) containing the lung 14. The isolated avian lung 14 is prepared as described in this disclosure. For example, the avian lung 14 may be decellularized and then recellularized with mammalian (human) cells. The housing 12 may be made from any suitable biocompatible material or combination of materials, including rigid materials and/or pliable materials. For example, the housing 12 may be made from polylactide (PLA), polyglycolide (PGA), polycaprolactone (PCL) and/or their copolymers, or other materials or combinations. Embodiments of the device 10 may be configured to be implanted in the body or configured for portable use outside of the body. The housing 12 may be manufactured using techniques such as 3D printing, injection molding, compression molding, and/or other manufacturing methods.

A blood supply conduit 20 is fluidically coupled to the pulmonary artery of the avian lung 14 and configured to be connected to a vessel of a patient 90. In this way, when coupled to a patient, a supply of deoxygenated blood may be provided from the patient 90 to the avian lung 14 via the blood supply conduit 20. A blood return conduit 22 is fluidically coupled to the pulmonary vein of the avian lung 14 and configured to be connected to an artery of a patient 90. In this way, when coupled to a patient, a supply of re-oxygenated blood may be provided from the avian lung 14 to the patient 90 via the blood return conduit 20. It should be noted that in embodiments in which the device is configured for implantation, the blood supply and/or return conduits may be formed, or partially formed, using the patient's own vasculature.

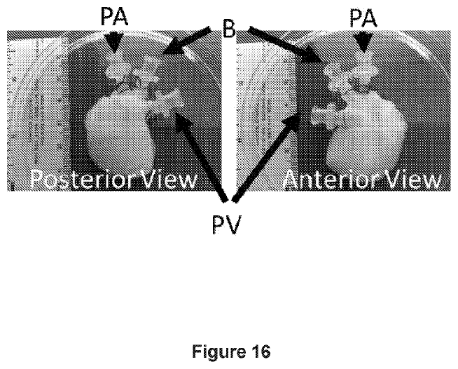

Gas flow through the decellularized or recellularized bird lung is unidirectional with two major bronchi cannulated—one for air/gas intake and one for air/gas egress. An example of one cannulated parabronchus is depicted in FIG. 16. A breathing gas conduit 24 is pneumatically coupled to a gas intake of the avian lung and configured to be connected to a source of breathing gas. The source of breathing gas may be, for example, a pressurized, bottled gas source, such as an air bottle or oxygen bottle, the ambient atmosphere in which the device is placed, a hospital breathing gas supply circuit, etc. An exhaust conduit 26 is pneumatically coupled to a gas egress of the avian lung for exhausting the spent breathing gas. As such, breathing gas is provided to the avian lung 14 by way of the breathing gas conduit 24 and is exhausted away from the avian lung 14 by way of the exhaust conduit 26. It should be noted that in embodiments in which the device is configured for implantation, the breathing gas and/or exhaust conduits may be formed, or partially formed, using the patient's own airway and/or structures of the patient's lung.

Multiple devices may be used simultaneously, and the multiple devices may be arranged in series and/or parallel with respect to the blood flow pathway. In other words, in a series arrangement, blood may flow from the patient to a first device and from the first device to a second device before returning to the patient, and in a parallel arrangement, blood from the patient separately to each device and then will flow from the respective device back to the patient. Similarly, a device may be connected in series and/or parallel with a patient's lung (where the device is used to supplement the patient's lung). For example, in a series arrangement, blood flow through the patient's lung before and/or after flowing through the device (or more than one device) and then flowing back to the patient's vasculature (e.g., non-pulmonary vasculature). In an exemplary parallel arrangement, blood flows separately through the patient's lung and the device in each case returning to the patient's vasculature (e.g., non-pulmonary vasculature).

Similarly, the multiple devices may be arranged in series and/or parallel with respect to the flow of breathing gas. The device (or multiple devices) and the patient's lung may be arranged in series and/or parallel with respect to the flow of breathing gas (with accommodations for the bidirectional flow of breathing gas in a mammalian lung). Furthermore, the blood flow arrangement may be the same or different from the breathing gas flow arrangement. For example, a first device may be in series with a second device with respect to the flow of blood, and the first device may be in parallel with the second device with respect to the flow of breathing gas.

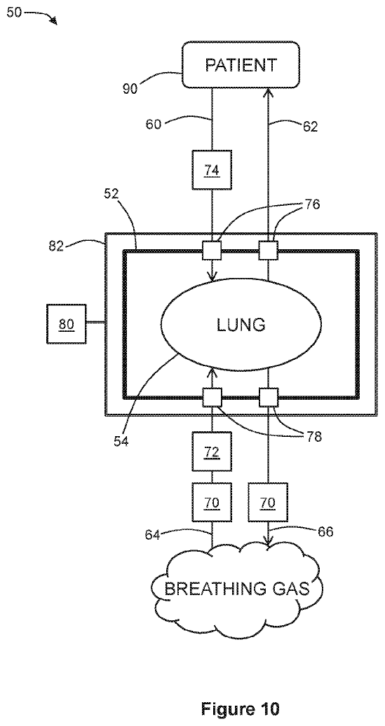

FIG. 10 is a more detailed depiction of an exemplary embodiment of a device 50 for lung supplementation or replacement. The avian lung 54 is housed within housing 52, and the housing includes a heating circuit 82 coupled to a controller 80. In this way, the temperature of the lung 54 may be regulated. In device 50, the blood supply conduit 60 includes a pump 74 to move blood from the patient 90 to the avian lung 54. Valves 76 are provided in the blood supply conduit 60 and the blood return conduit 62. Suitable valves 76 may be, for example, manually actuated valves for regulating the flow of blood and/or check valves for ensuring proper flow direction.

The breathing gas conduit 64 may include a gas pump 72 for moving breathing gas into the avian lung 54. In embodiments where the breathing gas is provided from a pressurized source, a pressure regulator may be provided in addition to or in place of the gas pump. Gas valves 76 are provided in the breathing gas conduit 64 and the exhaust conduit 66. Suitable valves 78 may be, for example, manually actuated valves for regulating the flow of breathing gas and/or check valves for ensuring proper flow direction. Each of the breathing gas conduit 64 and the exhaust conduit 66 also includes a filter 70 so as to, for example, capture contaminates in the gas flow.

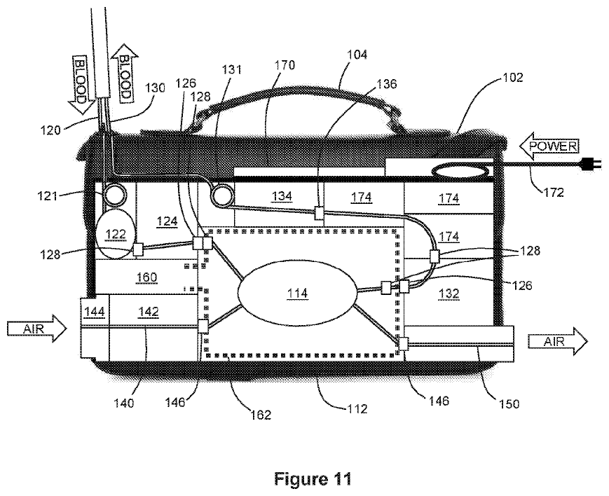

FIG. 11 is a diagram of another exemplary device 100 of the present disclosure, wherein device 100 is configured as a portable device contained in a carrying case 102 having a handle 104. Lung 114 is encased within housing 112. A heater 160 and/or heating circuit 162 are provided in order to maintain an acceptable temperature of the lung 114.

Blood supply conduit 120 is coupled to the pulmonary artery of the lung 114. A retractable spool 121 is provided such that the blood supply conduit 120 may be extended or retracted from the case 102 as needed. A bladder 122 is provided to provide a buffer to more readily maintain a steady supply of blood. A pump 124 moves blood through the blood supply conduit 120 to the lung 114. A valve 126 is provided in the blood supply conduit 120 between the pump 124 and the vessel 112. Flow sensors 128 are provided at one or more locations in the blood supply circuit to monitor blood flow. For example, flow sensors 128 may be provided before and after the valve 126. In this way, valves, such as valve 126 may be automatically adjusted (for example, by a microcontroller, etc.) based on a flow rate detected by the flow sensors 128.

A blood return conduit 130 is coupled to the pulmonary vein of the lung 114. A retractable spool 131 is provided such that the blood return conduit 130 may be extended or retracted from the case 102 as needed. A pump 132 is provided to urge blood flow from the lung 114 through the blood return conduit 130. A valve 126 is disposed in the fluid path of the blood return conduit 126 to regulate flow. As above, flow sensors 128 are provided at one or more locations in the blood return circuit to monitor blood flow. For example, flow sensors 128 may be provided before and after the valve 126. In this way, valves, such as valve 126 may be automatically adjusted (for example, by a microcontroller, etc.) based on a flow rate detected by the flow sensors 128. A pulse oximeter 134 is located along the blood return conduit 130 and a gas sensor 136 is disposed in the flow path of the blood return conduit 130. The pulse oximeter 134 and gas sensor 136 are configured to measure parameters including the percentage of oxygen saturation in the blood return conduit 130.

A breathing gas conduit 140 pneumatically connects a breathing gas supply with an air inlet of the lung 114. In this embodiment, the breathing gas source may be either ambient air or oxygen provided by an oxygen source (tank). A gas pump 142 may be used to draw air through a filter 144 and into the lung 114. A gas valve 146 is located in the flow path of the breathing gas conduit 140 to regulate the air flow into the lung 114. An exhaust conduit 150 is coupled to the lung 114 to exhaust used breathing gas. A gas valve 146 is provided to regulate the flow of breathing gas from the lung 114 to the atmosphere.

An operator can interact with the device 100 by way of a user interface 170. The user interface 170 may be located at, for example, the top of the device 100 under a flap of the case 102. In this way, the operator can monitor and/or adjust the flow of blood into and out of the lung 114, the oxygen present in the blood, the gas flow rate, etc. One or more batteries 174 are used to provide power to the various components of the device 100. The batteries 174 are charged by, for example, connection to a power supply via a power cord 172. Batteries 174 may be configured as primary and backup or other fault tolerant configurations which will be apparent to one having skill in the art in light of the present disclosure.

In contrast to the human tidal mechanism of diaphragm-based, bidirectional ventilation, the present device uses a avian architecture with unidirectional air flow. Therefore, decellularized and recellularized avian lungs of the present disclosure may be used as extracorporeal gas exchange devices utilizing unidirectional air flow. If used as an implant within a human body, the decellularized and recellularized avian lungs can be used as a supplement to a host's lungs. When used in the extracorporeal setting, the decellularized and recellularized avian lungs can be used as an alternative to the host's lungs or to augment a host lung function.

Decellularized avian lungs may be used as vectors for whole lung bioengineering and may help ameliorate a shortage of donor lungs needed for a growing population with severe lung disease and organ failure.

### Example 1

In this example, using a modification of the detergent-based techniques for decellularizing mammalian lungs, intact scaffolds were produced and assessed by a range of histologic, immunohistochemical, and mass spectrometric assessments. The presence of preserved collagen type I, collagen type IV, elastin, laminin, and fibronectin in the decellularized chicken lungs provides a similar framework to which a range of differentiated human lung epithelial, stromal, and pulmonary vascular endothelial cells or lung progenitor cells can adhere.

Methods

Avian Lungs:

Chicken (Gallus gallus domesticus) and emu (Dromaius novaehollandiae) lungs were procured post mortem from local farms/slaughterhouses. A total of 14 chicken and 7 emu lungs were studied.

Healthy chickens were euthanized via a standard method of slitting the throat and exsanguination at a local farm, and the carcass was immediately cooled on ice and transported to the laboratory for dissection and extraction of the heart-lung-trachea bloc via a bilateral dorsal thoracotomy approach as follows: the trachea was initially identified and isolated in the neck, separated from the esophagus, and cannulated with a tubing connector (inner diameter 5/32″ inch and a Luer lock female ending, Cole-Parmer) which was secured in place via silk suture. The dorsum of the chicken was identified and the bilateral scapulae were mobilized superficially from the rib cage below. The thorax of the bird was then entered bilaterally rostral to the lung, with care not to damage underlying structures. The lungs and air sacs were identified and using blunt dissection, carefully peeled the lungs from the costal structures, leaving the air sacs in situ. Anteriorly, the trachea was followed rostrally until the heart and lung structures were identified. The trachea, heart, and lungs were then separated and removed en bloc. The pulmonary arteries were identified bilaterally and preserved, as was the entirety of the tracheobronchial tree. If upon flushing of the trachea an air sac ostium was identified, it was ligated with a small surgical clip or suture. The pulmonary arteries were each identified and individually cannulated with 18 gauge needles bilaterally.

Healthy emus being processed for commercial use were euthanized under standard protocol at a local farm. Lungs were identified, isolated, and preserved by the farm personnel in collaboration with the research team. Samples were preserved on ice and transported to the laboratory for immediate decellularization. Emu lungs were cannulated via their main bronchus and pulmonary arteries in a similar fashion as the chickens. Given the larger size of the emu lungs, two approaches were utilized: in some lungs, both lungs were processed together, in others, the right and left lungs were separated and processed individually.

Injection Molding:

Chickens were euthanized and their tracheae were cannulated. The rest of the avian physiology was left intact in order to not disturb and prevent rupture of air sacs. Oomoo® 30 silicone solution (Smooth-On, Inc., Macungie, Pa., USA) was obtained and utilized as per manufacturer's instructions. In order to reduce its viscosity, kerosene 1-K Heater Fuel (Klean Strip, Memphis, Tenn., USA) was added at a 1:8 ratio to the current silicone solution. Using a Luer lock 60 mL syringe, the silicone was injected via the cannulated trachea into the native tissue. The chicken was held so that the trachea was the highest point in the system, allowing air to escape during the solution instillation. Once the airways were filled with 300 mL of solution, the approximate volume of the chicken airway, the syringe was left locked to the trachea to maintain inflation pressure. After overnight incubation, tissue was removed mechanically using surgical tools. The mold was then submerged in a 1 M hydrogen sulfate [HSO₄⁻] solution overnight. If tissue had not cleared, the solution was replaced and the mold was submerged again overnight. After the isolation was complete, the mold was submerged in 70% (v/v) ethanol overnight for disinfection.

Lung Decellularization:

Extracted lungs were stored on ice and transported for immediate processing. After anatomic identification of major artery and airways (either trachea or major bronchi), each lung was thoroughly flushed with deionized water (DI) containing heparin sulfate (1U/ml, Fisher Scientific, Waltham, Mass., USA) to clear all blood. Each lung (emu), or set of lungs (chicken) was perfused with the following detergents and membrane-destabilizing solutions in a sequentially fashioned protocol using a roller pump (Stockert Shiley) with a flow rate of 2 L/min. Under sterile conditions, each lung was flushed with 4 L of DI solution, followed by 4 L of 0.1% Triton X-100 (Sigma-Aldrich, St. Louis, Mo., USA) solution per lung and kept in 0.1% Triton X-100 for 24 hours at 4° C. on a shaker. Lungs were again flushed with 4 L DI, followed by 4 L of 2% sodium deoxycholate (SDC, Sigma-Aldrich) per lung and kept in 2% SDC for 24 hours at 4° C. on a shaker. Lungs were again flushed with 4 L DI, followed by 4 L of 1 M sodium chloride (NaCl, Sigma-Aldrich) and kept in 1M NaCl solution for 1 hour at room temperature (RT) on a shaker before flushing them with 4 L DI. After flushing with 4 L DNAse solution (30 mg/L bovine pancreatic deoxyribonuclease, 1.3 mM MgSO₄, 2 mM CaCl₂) in DI water, all Sigma Aldrich) per lung, lungs were kept in DNAse solution for 1 hour at RT on a shaker. After again flushing with 4 L DI, lungs were flushed with 4 L 0.1% peracetic acid (Sigma-Aldrich) in 4% ethanol per lung and kept in 0.1% peracetic acid solution at RT for one hour on a shaker. Afterwards lungs were flushed with PBS storage solution: 1× Phosphate Buffered Saline Solution (Corning, Corning, N.Y., USA) supplemented with Penicillin/Streptomycin (500 IU/mL Penicillin/500 μg/mL Streptomycin, Lonza, Basel, Switzerland), Gentamicin (50 mg/L, Corning), and Amphotericin B (2.5 mg/L, Corning). At the conclusion of the decellularization, samples/biopsies were procured and the samples were stored in storage solution at 4° C. until further processing or usage for reseeding. Lungs were stored for up to 3 months.

In comparison to mammalian lungs, bird lungs work through a cross current system. As such, each step of the perfusion decellularization was performed utilizing a continuous loop perfusion pump (as opposed to intermittent filling) to maximize lung filling and detergent efficacy. This was accompanied by manual manipulation (intermittent tissue massage) comparable to use of manual manipulation of the tissue after each filling for decellularization of mammalian lungs. Perfusion was performed at a rate of 2 L/min for chicken and 3 L/min for emu lungs for a total of 10 minutes on-pump with the respective solution. These flow rates were designed to maintain a full, static volume within the lung, with distention but not disruption of tissue which was confirmed afterwards by histology and transmission electron microscopy. At completion of on-pump perfusion the organs were transferred to their respective solutions on a shaker for further decellularization.

Assessment of Residual DNA:

Native and decellularized lung tissue was dried on a tissue paper (Kimwipe, Kimtech, Kimberly-Clark, Roswell, Ga., USA) until no liquid was visibly seen to be released from it, weighed, and DNA was extracted using the DNeasy Blood & Tissue Kit (Qiagen, Hilden, Germany) following the instructions provided by the manufacturer. The isolated DNA was run on a 0.8% agarose gel and visualized under UV light with SYBR Safe DNA Gel stain (Invitrogen, Carlsbad, Calif., USA) using the Versa Doc (BioRad, Hercules, Calif., USA). A 100 bp ladder and salmon sperm DNA (Invitrogen) was used as DNA size marker and positive control. DNA was quantified using a Nanodrop (Thermo Scientific) and threshold for adequate decellularization was set at less than 50 ng/mg dry tissue weight.

Assessment of Residual Detergent:

Concentrations of SDC in wash effluents were determined using a modified methylene blue (MB). In short: effluent samples were mixed with 0.0125% MB (Sigma-Aldrich) in DI water (w/v) at a ratio of 1:10. After vortexing the samples with MB, chloroform (Sigma-Aldrich) was added at a ratio of 1:2 (sample: chloroform, v/v). Samples were then vortexed for 1 min. Following a 30 minute incubation period at RT, 150 μl of the bottom chloroform layer was extracted and the absorbance at 630 nanometers (nm) was measured in a Synergy HT Multi-Detection Microplate Reader (Biotek Instruments, Winooski, Vt., USA) in a polypropylene 96-well plate (Costar, Corning, N.Y., USA). Pure DI-water or PBS (Mediatech Inc., Manassas, Va., USA) containing no detergents served as the blank. SDC concentration was calculated based on SDC standard curves prepared in either DI water (for Triton, SDC, NaCl, and DNAse effluents) or storage solution (for PBS effluents).

Lung Histology:

Decellularized lungs were fixed by submersion in 4% paraformaldehyde for at least 3 hours at room temperature, embedded in paraffin, and 5-μm sections mounted on glass slides. Following deparaffinization, sections were stained with hematoxylin & eosin, Verhoeff's Van Giesson (EVG), Masson's Trichrome, or Alcian Blue, and were assessed by brightfield light microscopy.

Electron Microscopy:

For electron microscopic analyses, segments of decellularized chicken and emu lungs were fixed overnight at 4° C. in Karnovsky's fixative (2.5% glutaraldehyde, 1.0% paraformaldehyde in 0.1M Cacodylate buffer, pH 7.2). After rinsing in Cacodylate buffer, the tissue was minced into 1 mm³ pieces and then fixed in 1% osmium tetroxide for 2 hours at 4° C. Subsequently, the pieces were rinsed again in Cacodylate buffer, dehydrated through graded ethanols, then cleared in propylene oxide, and embedded in Spurr's epoxy resin (all reagents from Electron Microscopy Sciences, Hatfield, Pa., USA). Semi-thin sections (1 μm) were cut with glass knives on a Reichert ultracut microtome (Reichert-Jung, Vienna, Austria), stained with methylene blue-azure II (Electron Microscopy Sciences) and then evaluated for areas of interest (proximal and distal alveolar septae, large/small airways, blood vessels). Ultrathin sections (60-80 nm) were cut with a diamond knife, retrieved onto 200 mesh thin bar nickel grids (Electron Microscopy Sciences), contrasted with uranyl acetate (2% in 50% ethanol, Electron Microscopy Sciences) and lead citrate (Electron Microscopy Sciences), and examined with a JEOL 1400 TEM (JEOL USA, Inc, Peabody, Mass., USA) operating at 60 kV.

Immunohistochemical (IHC) Staining:

Standard deparaffinization was performed with three separate 10 min incubations in xylenes (Fisher Scientific), followed by rehydration in a descending series of ethanols, and finally in water. Antigen retrieval was performed by heating tissue in 1× sodium citrate buffer (Dako, Carpentaria, Calif., USA) at 98° C. for 20 minutes followed by a brief 20 minutes cool at room temperature. Tissue sections were permeabilized in 0.1% Triton X-100 solution for 15 minutes. Triton X-100 was removed with two 10 minute washes in 1% BSA (Sigma) solution. Blocking was performed with 10% goat serum (Jackson Immuno Research, West Grove, Pa., USA) for 60 minutes. After blocking, primary antibody was added and tissue sections were incubated overnight at 4° C. in a humidified chamber. Tissues were washed three times with 1% BSA solution for 5 minutes each. Secondary antibody was added and incubated for 60 min at room temperature in a dark humidified chamber. Tissues were again washed three times in 1% BSA solution for 5 minutes each in the dark. DAPI nuclear stain (Invitrogen/Life Technologies/Thermo Fisher) was added for 5 minutes at room temperature in the dark followed by 2 washes in 1% BSA solution for 5 minutes each. The sections were finally mounted in Aqua Polymount (Lerner Laboratories, Pittsburgh, Pa., USA).

As there are limited antibodies available specifically against bird proteins, we utilized those available and when not available, utilized commercially available antibodies for mammalian proteins. As detailed in the results section, we were able to validate reactivity of each antibody utilized with the respective bird proteins. Primary antibodies used were: purified mouse anti-fibronectin monoclonal (610077—1:100—BD Transduction Laboratories, Franklin Lakes, N.J., USA), laminin antibody polyclonal (ab11575—1:100—Abcam, Cambridge, United Kingdom), rabbit polyclonal to alpha elastin (ab21607—1:100—Abcam), smooth muscle myosin heavy chain 2 polyclonal (ab53219—1:100—Abcam), collagen I polyclonal (ab292—1:100—Abcam), Ki67 proliferation marker polyclonal (ab16667—1:50—Abcam), cleaved caspase-3 polyclonal (Asp175—1:100—Cell Signaling Technology, Danvers, Mass., USA), mouse clone anti-human actin polyclonal (1A4—1:10,000—Dako via FAHC, Denmark). Secondary antibodies used: Alexa Fluor 568 goat anti-rabbit IgG (H+L) (1:500, Invitrogen), Alexa Fluor 568 F(ab′)2 fragment of goat anti-mouse IgG (H+L) (1:500, Invitrogen).

Mass Spectrometry:

Three samples of about 125 mg wet weight from different locations of each decellularized lung tissue were procured. Each sample was homogenized using a Polytron PT2100 (Kinematica, Luzern, Switzerland) in 200 μl of 4× lysis buffer (250 mM Tris pH 6.8, Sigma, 8% SDS, BioRad, 400 mM DTT, Sigma, 40% Glycerol, Sigma) and diluted with DI water to 1×. After centrifugation for 5 min at 15,000 g at 4° C. supernatant and pellet were separated. Protein content of the supernatant was evaluated with the DC detergent compatible protein detection Kit (BioRad). 20 μg of protein were loaded onto a 10% SDS PAGE gel and individual bands containing chicken or emu proteins were excised and prepared for mass spectrometry using a standard in-gel trypsin digestion protocol (Tacoma et al., J Proteomics. 2016; 130:200-10). Briefly, gel bands were cut into 1-mm³ pieces and destained overnight using 50 mM ammonium bicarbonate in 50% acetonitrile. After reduction by 10 mM dithiothreitol (DTT) at 55° C. for 1 hour the gel pieces were alkylated with 55 mM iodoacetamide (IAA) in the dark at room temperature for 45 min. The gel pieces were then washed and dehydrated twice alternately with 100 mM ammonium bicarbonate and 100% acetonitrile (ACN). The gel pieces were dried in a SpeedVac (Thermo Savant, Waltham, Mass., USA) and then subjected to trypsin digestion using sequencing grade trypsin (Promega, Madison, Wis., USA) for 17 hours at 37° C. The tryptic digests were acidified with 150 μl of 5% formic acid (FA) in 50% acetonitrile to stop the reaction. The peptides were extracted, dried and kept in a −80° C. freezer until they were analyzed by mass spectrometry.

The dried digests were re-suspended in 50 μl of 2.5 ACN/2.5% FA in water and 5 μl of sample was loaded onto a capillary fused silica column (12 cm×100 μm inner diameter) packed with HALO C18 (2.7 μm particle size, 90 A, Michrom Bioresources, CA, USA) and run at a flow rate of 300 nL/min. Peptides were separated by a gradient of 0-35% ACN/0.1% FA (Fisher Chemical, Optima, LC/MS grade) over 120 min, 35-100% ACN/0.1% FA for 1 min, and a hold of 100% ACN for 8 min, followed by an equilibration 0.1% FA in H₂O for 21 min. Peptides were introduced to the Q-Exactive mass spectrometer (Thermo Fisher Scientific, Waltham, Mass., USA) via a nanospray ionization source and a laser pulled ˜3 μm orifice with a spray voltage of 2.2 kV. Mass spectrometry data were acquired in a data-dependent “Top 10” acquisition mode, in which a survey scan from m/z 360-1600 was followed by 10 higher-energy collisional dissociation (HCD) tandem mass spectrometry (MS/MS) scans of the most abundant ions. MS/MS scans were acquired with the following parameters: isolation width=1.6 m/z, normalized collision energy=26.

Product ion spectra were searched using the SEQUEST HT engine implemented on the Proteome Discoverer 1.4 (Thermo Fisher Scientific, Waltham, Mass., USA) against a Uniprot Gallus gallus (UP000000539, May 14, 2016 release was downloaded) and Dromaius novaehollandiae from the NCBI protein database (May 14, 2016). Search parameters were as follows: (1) full trypsin enzymatic activity, (2) two missed cleavages, (3) peptides between the MW of 350-5000, (4) mass tolerance at 20 ppm for precursor ions and 0.02 Da for fragment ions, (5) Dynamic modifications on methionine (+15.9949 Da: oxidation), (6) 3 maximum dynamic modifications allowed per peptide; and (7) static modification on cysteine (+57.0215 Da: carbamidomethylation). The combined data set was filtered to contain less than 1% false positive (with the Target Decoy PSM Validator node).

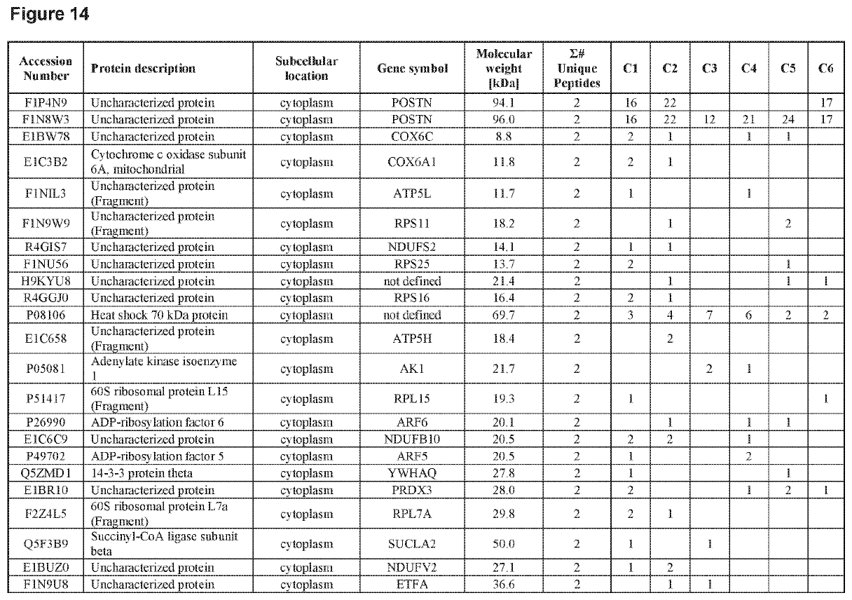

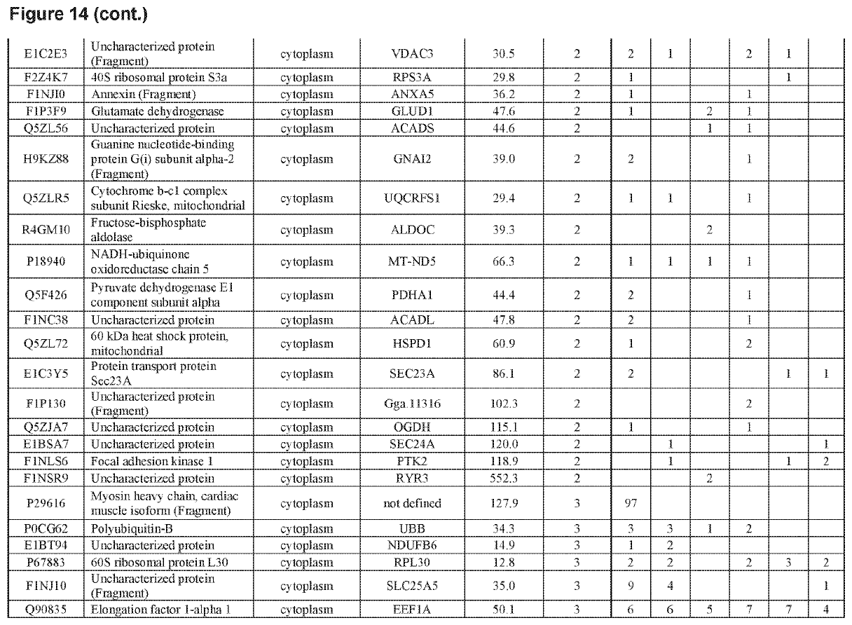

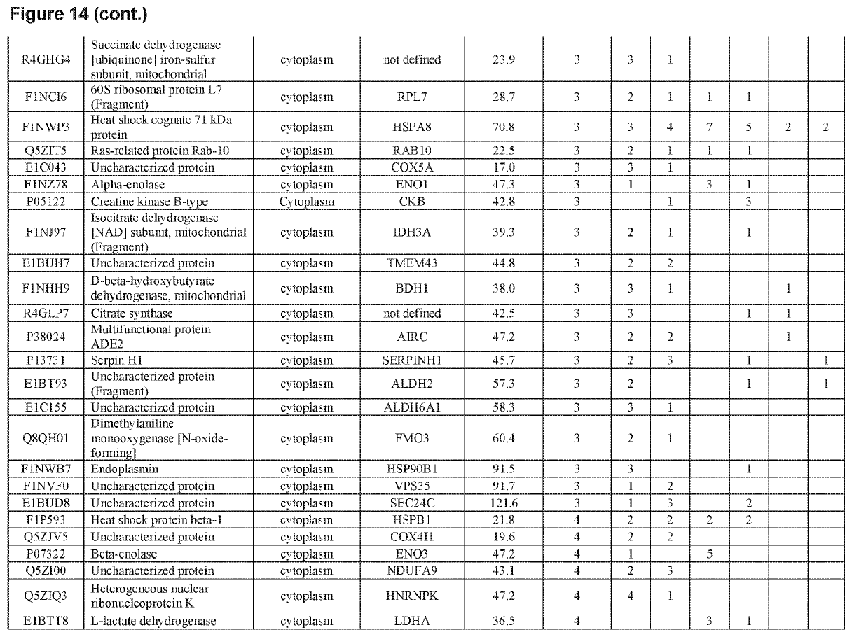

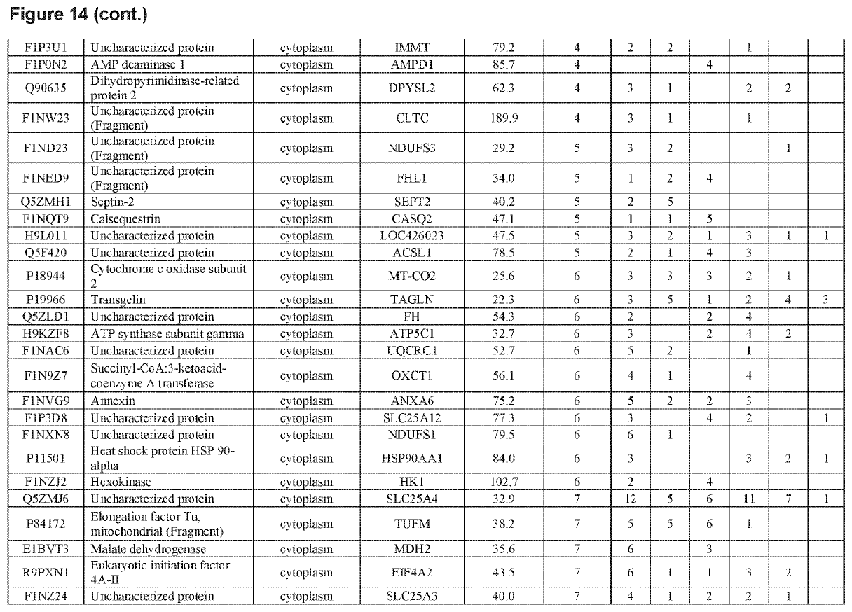

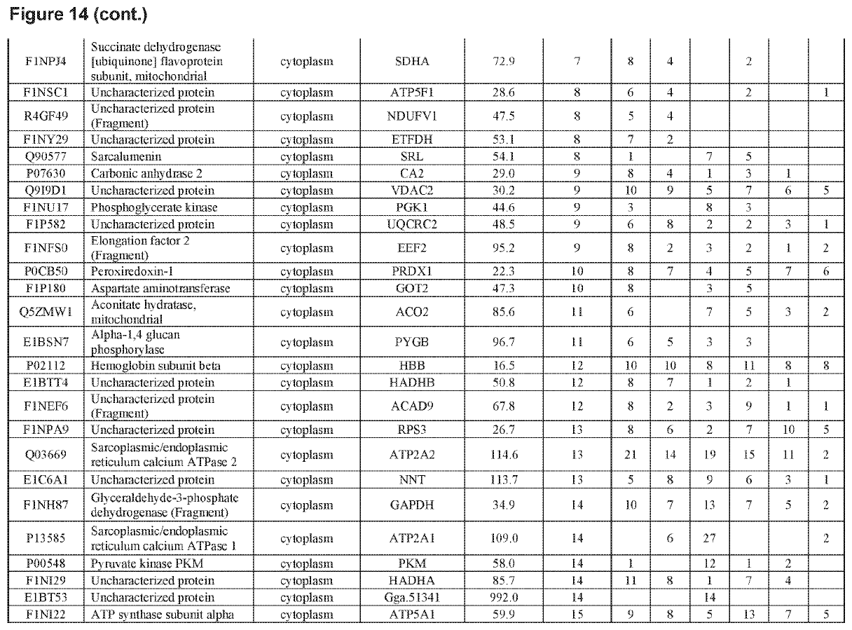

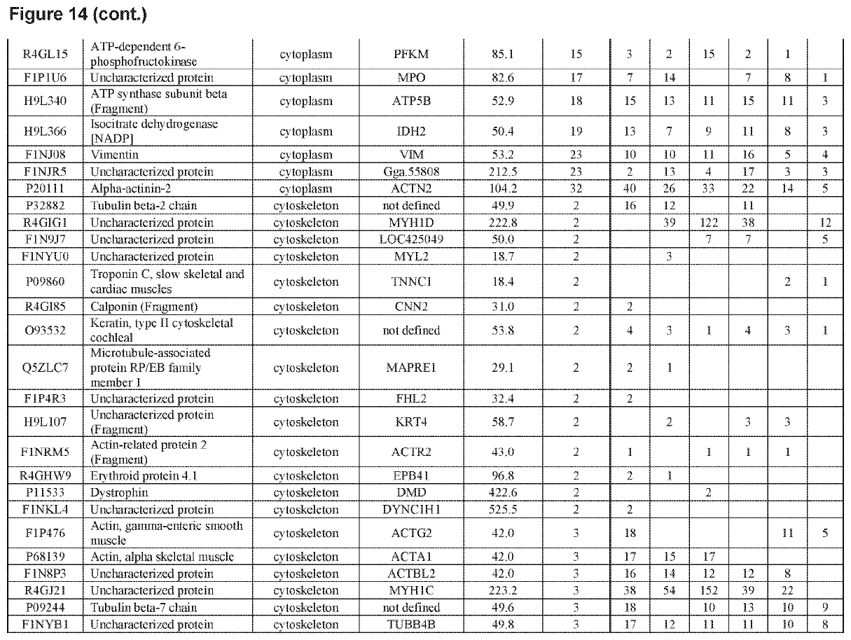

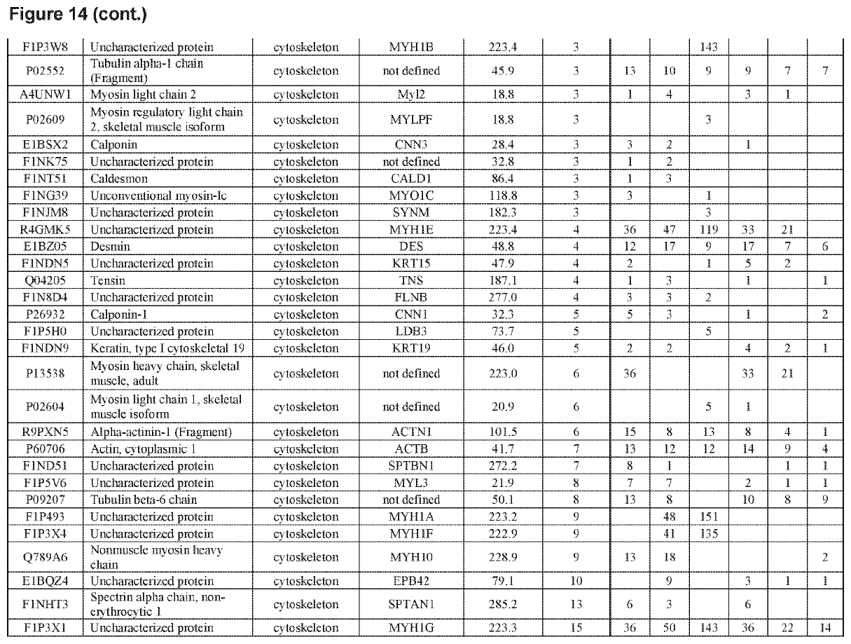

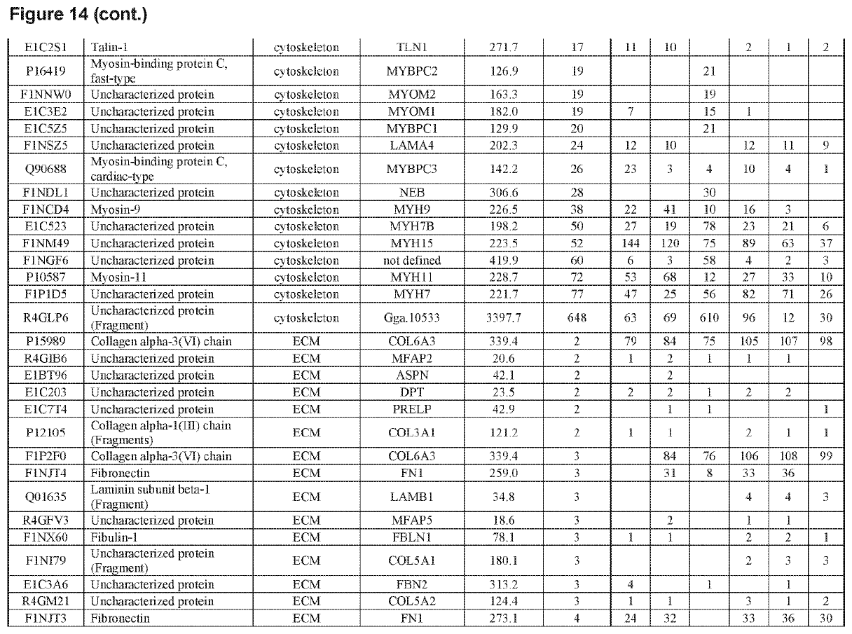

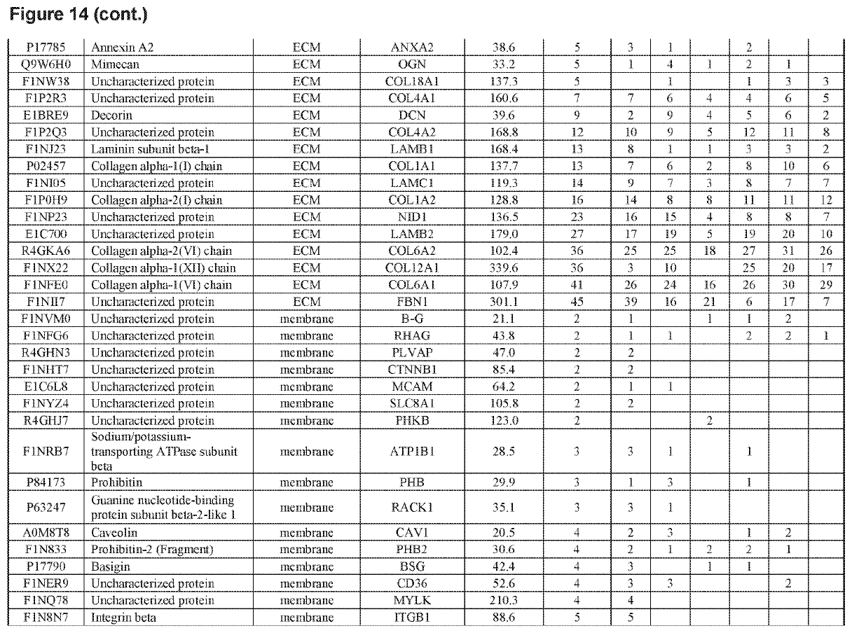

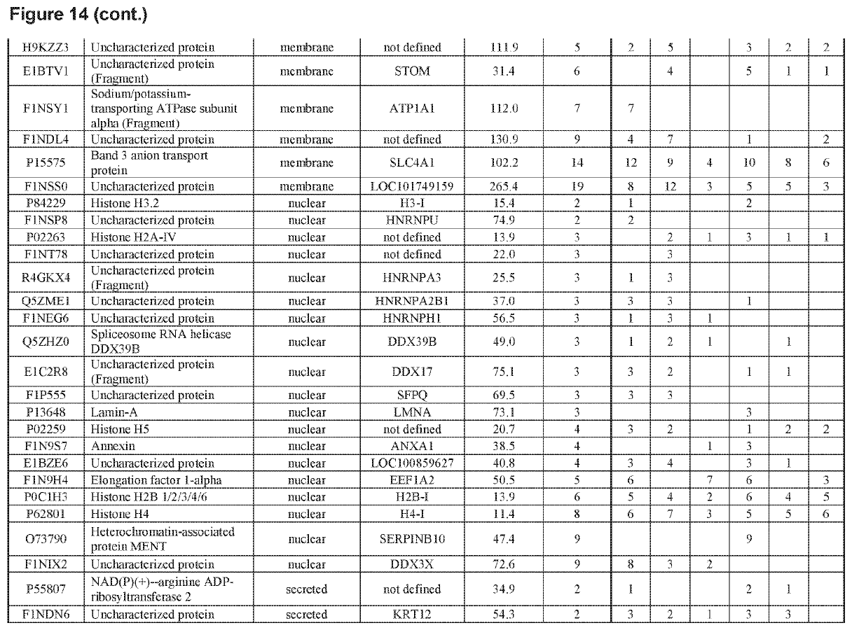

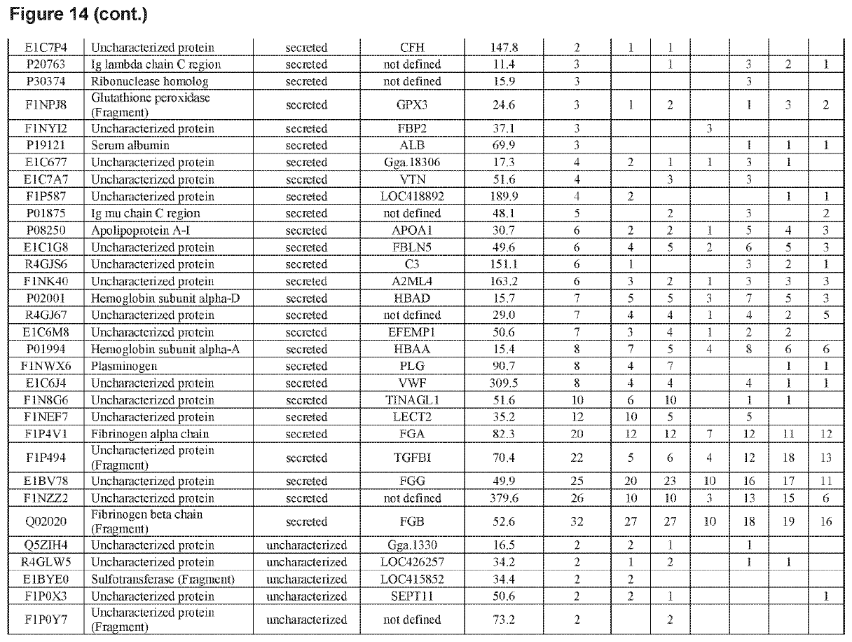

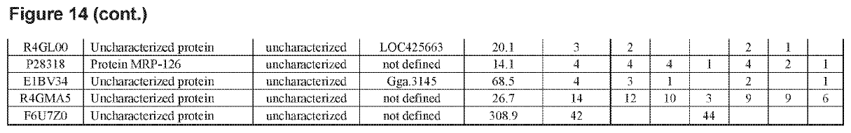

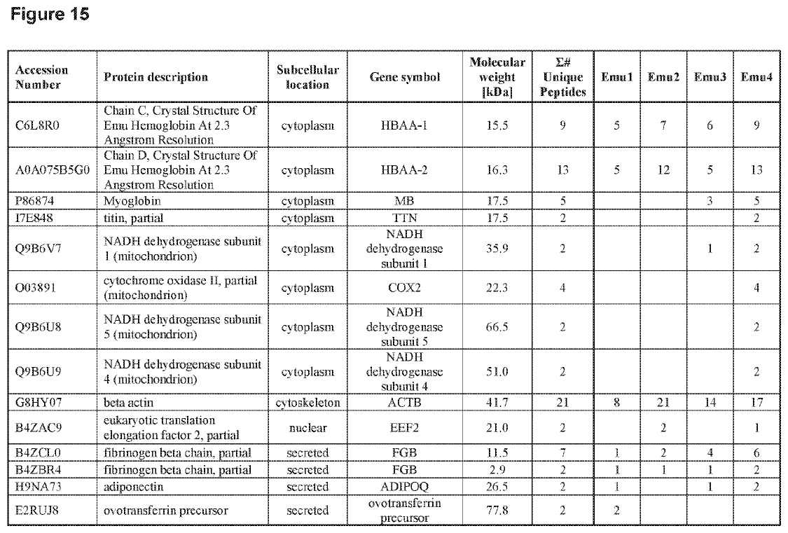

Proteins positively identified with two or more distinct peptide hits were assigned to one of six groups: ECM, cytoplasm, cytoskeletal, nuclear, membrane-associated, secreted, and uncharacterized in case no subcellular location was specified. Heatmaps were generated with the log 2 transformation of peptide hits from each positively identified protein with clustering of the rows to display genes that are similarly expressed. If any of the proteins were matched to more than one category, we chose its predominant subcellular location for functional grouping. In FIGS. 14 and 15, we also displayed single peptide hits.

Preparation and Culture of Recellularized Chicken Lungs and Emu Segments:

Whole chicken lungs or small, approximately 10-15 cm³ pieces of decellularized emu lungs excised from the larger lobes were used. Under sterile technique the largest corresponding bronchus or parabronchus of the lung/segment was cannulated with blunted 18.5 or 25G cannulas. After the cannulas were secured with titanium clips (Teleflex Medical, Wayne, Pa., USA) the lungs/segments were coated in 2.5% sodium alginate (Manugel, FMC Biopolymer, Philadelphia, Pa., USA) and then immediately cross-linked with a 3% calcium chloride (Sigma) solution, resulting in segments being uniformly coated in a calcium alginate hydrogel that serves as an artificial pleural coating. Hydrogel-coated lungs/segments were then inoculated with cell suspensions (4.5-5.0×10⁷ cells per lung/segment, suspended in 1.0 ml media) in the respective compartment (hMSC, HLF, HBE via airways and CBF via the vasculature) and allowed to incubate at 37° C. overnight to allow cellular attachment. The following day lungs/segments were sliced into approximately 1 mm thin sections with sterile razor blades and each slice placed in an individual well of a 24-well non-tissue culture treated dish, covered with 2 mL of sterile cell cultivation media, and placed in a standard tissue culture incubator at 37° C. with 5% CO² as previously described (Wagner et al., Biomaterials. 2014, 35:2664-79). Cell cultivation media was replaced routinely at 48 hour intervals. Slices were harvested at 1, 3, 7, 14, and 28 days post-inoculation and fixed for at least 4 hours at room temperature in 4% paraformaldehyde. Harvested samples were embedded in paraffin, cut, and mounted as 5 μm sections, and then assessed by H&E staining for the presence and distribution of the inoculated cells.

Cells and Cell Inoculation:

Human bronchial epithelial cells (HBE) were cultured on cell-culture treated plastic at 37° C. and 5% CO² in serum free culture medium consisting of DMEM/F-12 50/50 mix (Corning), 10 ng/ml Cholera toxin (Sigma), 10 ng/ml epidermal growth factor (Sigma), 5 μg/ml insulin (Gemini Bio-Products, West Sacramento, Calif., USA), 5 μg/ml transferrin (Sigma), 0.1 μM dexamethasone (Sigma), 15 μg/ml bovine pituitary extract (Sigma), 0.5 mg/ml bovine serum albumin (Life Technologies), and 100 IU/ml penicillin/100 μg/ml streptomycin (Corning). Human Lung Fibroblasts (HLF) (ATCC, CCL 171) were grown in media consisting of DMEM/F-12 50/50 mix (Corning), 10% fetal bovine serum (Hyclone), 100 IU/ml penicillin/100 μg/ml streptomycin, 2 mM L-glutamine (Corning). CBF (pulmonary endothelial colony forming cells) cells were grown in EGM-2 (Lonza) supplemented with 5% fetal bovine serum, 0.04% hydrocortisone, 0.4% hFGF-B, 0.1% VEGF, 0.1% R3-IGF-1, 0.1% ascorbic acid, 0.1% hEGF, 0.1% gentamicin sulfate amphotericin-B, and 100 IU/ml penicillin/100 μg/ml streptomycin. These cells were expanded on collagen type I coated tissue culture surfaces. Human bone marrow-derived mesenchymal stromal cells (hMSCs) were obtained from the University of Minnesota through the NHBLI Production Assistance for Cell Therapy program. These cells have previously been extensively characterized for cell-surface marker expression and differentiation capacity (Reed et al., Transfusion. 2009, 49:786-96). Cells were expanded in culture using media consisting of Modification of Eagle Medium-Earls Balanced Salt Solution (MEM-EBSS) (Hyclone, Thermo Scientific), 20% fetal bovine serum, 100 IU/ml penicillin/100 μg/ml streptomycin, 2 mM L-glutamine, and used only at no more than passage 7.

Statistical Analyses:

For mass spectrometry assessments, heatmaps for the log 2 of unique peptide hits for each positively identified protein in the mass spectrometric analyses of lungs decellularized under each experimental condition were generated using the ‘pheatmap’ package for ‘R’ statistical software version 2.15.1. Differences between Ki67 or caspase-3 expression were assessed by two way ANOVA with Bonferroni post-test.

Results

Decellularized Avian Lungs Qualitatively Maintain Extracellular Matrix Structure by Histologic and Immunohistochemical Evaluations with Maintenance of Key Extracellular Matrix Proteins Similarly Present in Decellularized Mammalian Lungs.

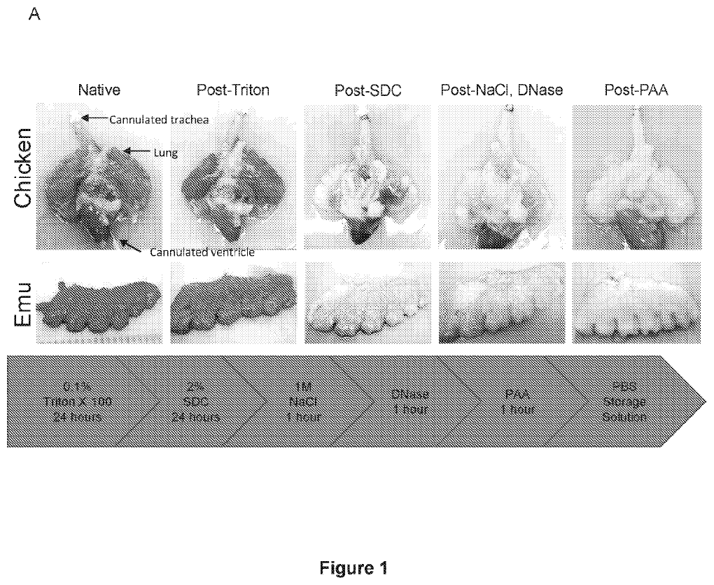

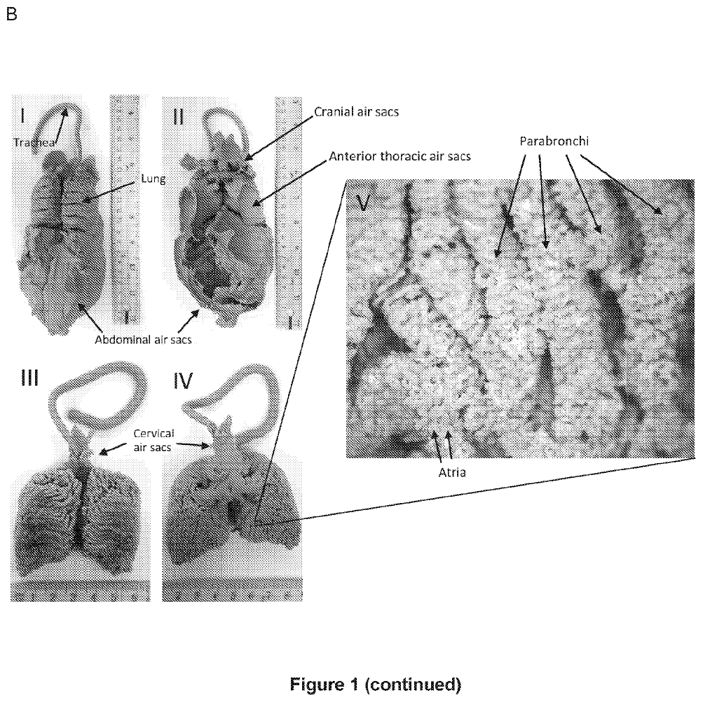

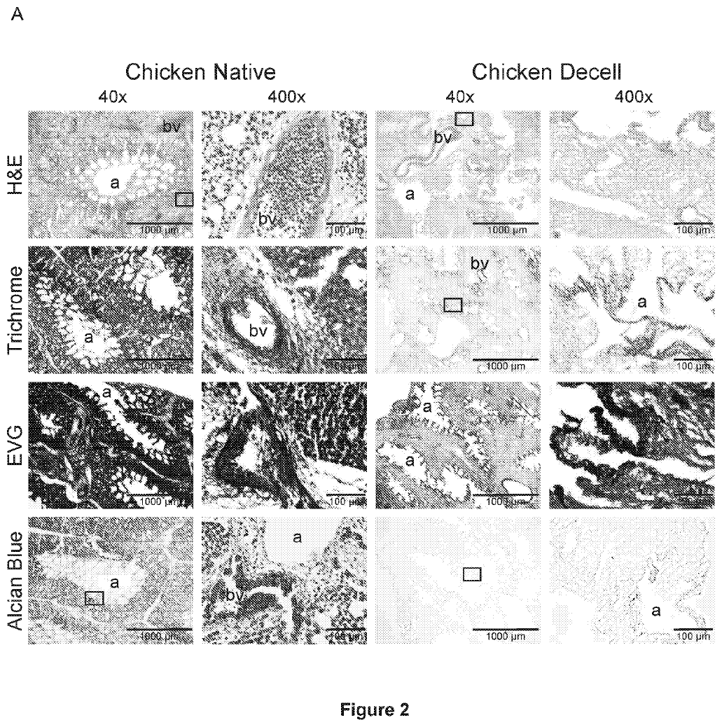

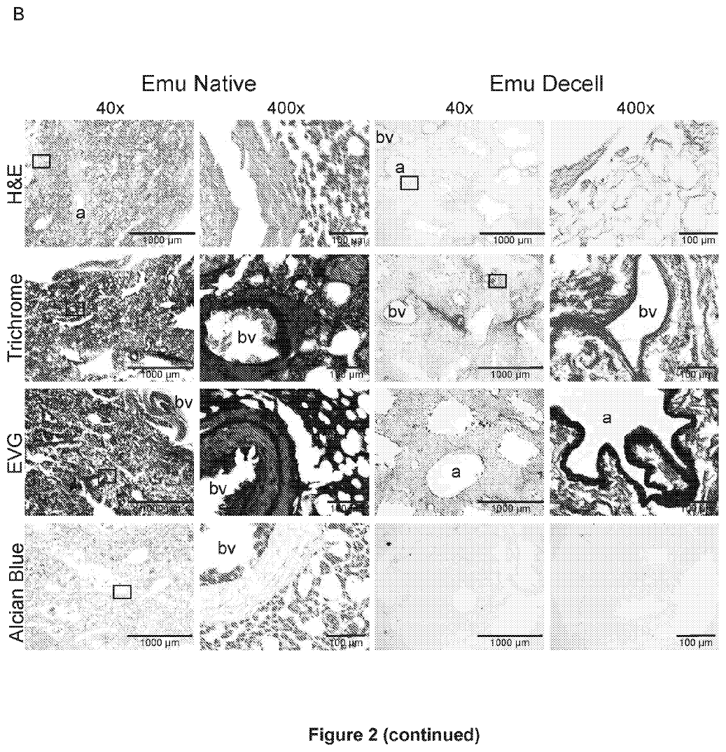

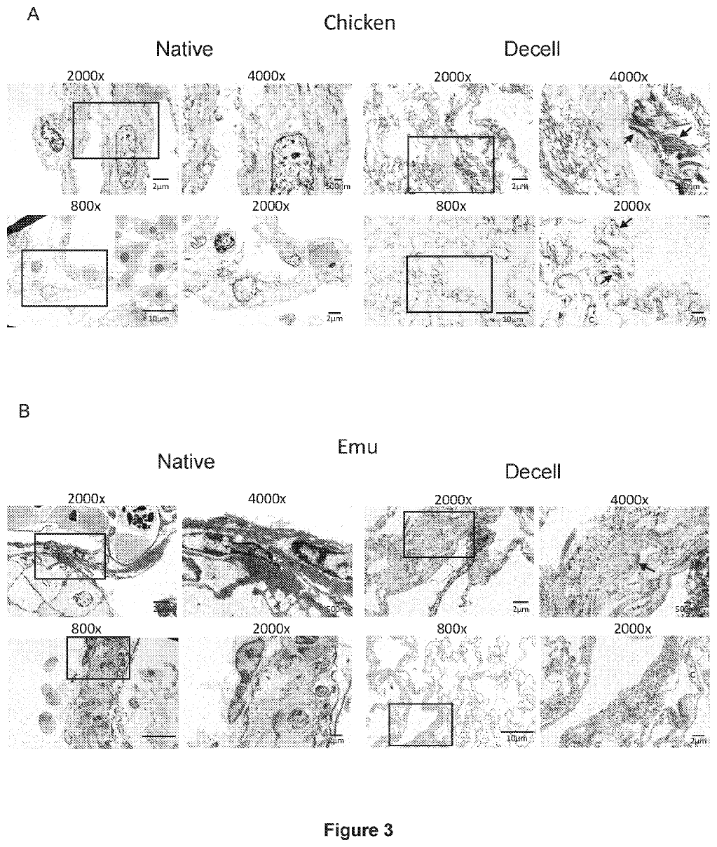

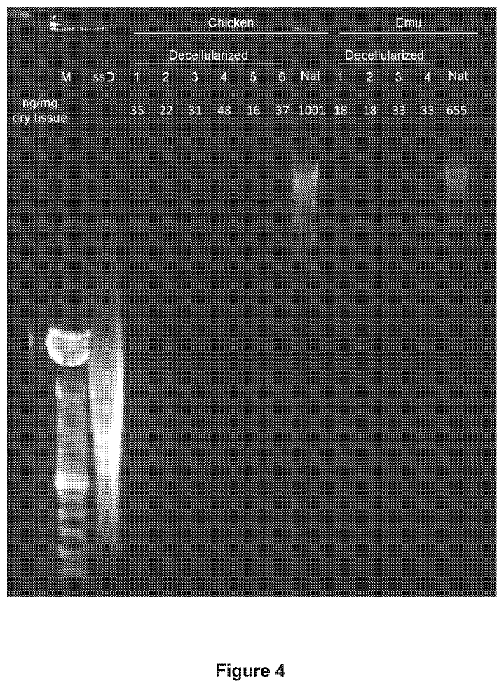

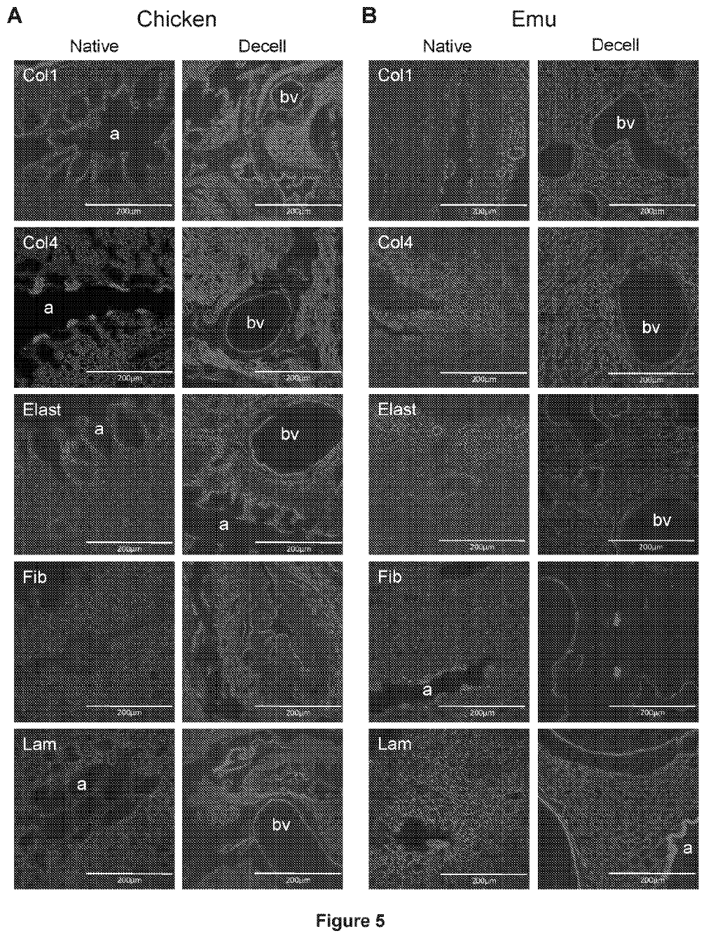

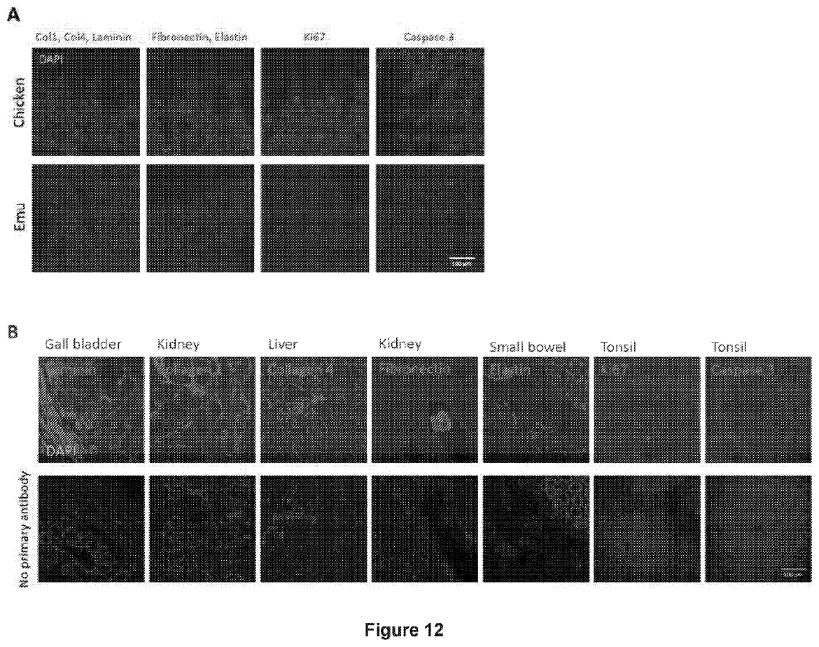

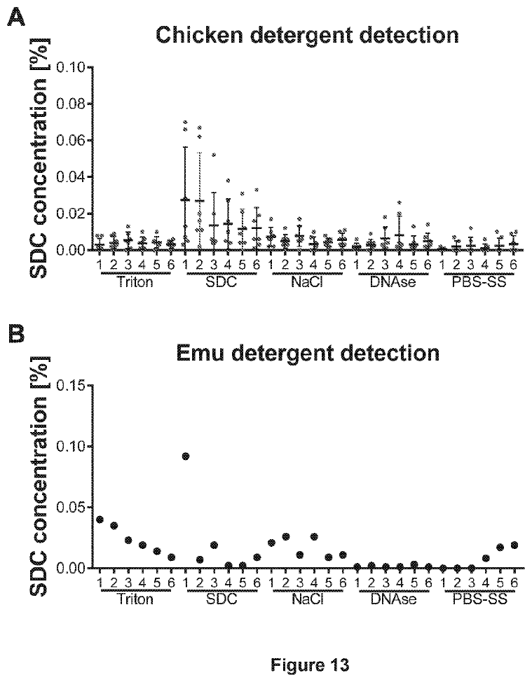

A Triton X-100/sodium deoxycholate (SDC) detergent-based decellularization protocol with constant flow perfusion (2 liters/minute) of both the vasculature and airways, a method previously optimized for use in mammalian lungs was re-optimized for use in avian lungs. Chicken and emu lungs underwent successful decellularization as demonstrated by the progressive loss of pink coloration leading to a final translucent pearly white gross appearance. Greater understanding of avian pulmonary anatomy of the decellularized lungs was gained by creating the injection molds of native chicken lungs. This allowed for more effective identification and ligation of air sacs prior to decellularization (FIG. 1). Overall microarchitecture of the lungs was preserved as observed in histologic stainings with hematoxylin and eosin (H&E), Verhoeff's Van Gieson (EVG), Masson's trichrome, and Alcian blue (FIG. 2). There was no residual cellular debris or cellular material detectable in either chicken or emu lungs. Similar to the decellularization of mammalian lungs (rodent, porcine, non-human primate, or human), a qualitative decrease in both elastin and in glycosaminoglycans was observed with EVG and Alcian blue staining, respectively. Electron microscopic evaluation demonstrated retention of characteristic ECM structures including collagen fibrils and intact capillaries (FIG. 3). DNA quantification in decellularized chicken and emu lungs demonstrated residual levels below 50 ng/mg and no residual fragments were observed on DNA gels, thereby suggesting adequate removal of nuclear material (FIG. 4). Immunofluorescence staining for specific ECM proteins demonstrated general qualitative retention of collagen I, collagen IV, and laminin, in both chicken and emu lungs while elastin and fibronectin seemed decreased in emu lungs only (FIG. 5). Notably, as many of the commercial antibodies utilized had not been previously validated in chicken and emu tissue, appropriate positive and negative controls demonstrated cross-reactivity with the bird lung ECM proteins (FIG. 12). Furthermore it is known that residual detergent in the decellularized lungs might affect cell viability and proliferation. Therefore, residual detergent (SDC) was assessed and was found to be below detectable or significant limits in both decellularized chicken and emu lungs (FIG. 13)

Mass Spectrometry Analysis of Decellularized Chicken and Emu Lungs is Limited by the Available Databases.

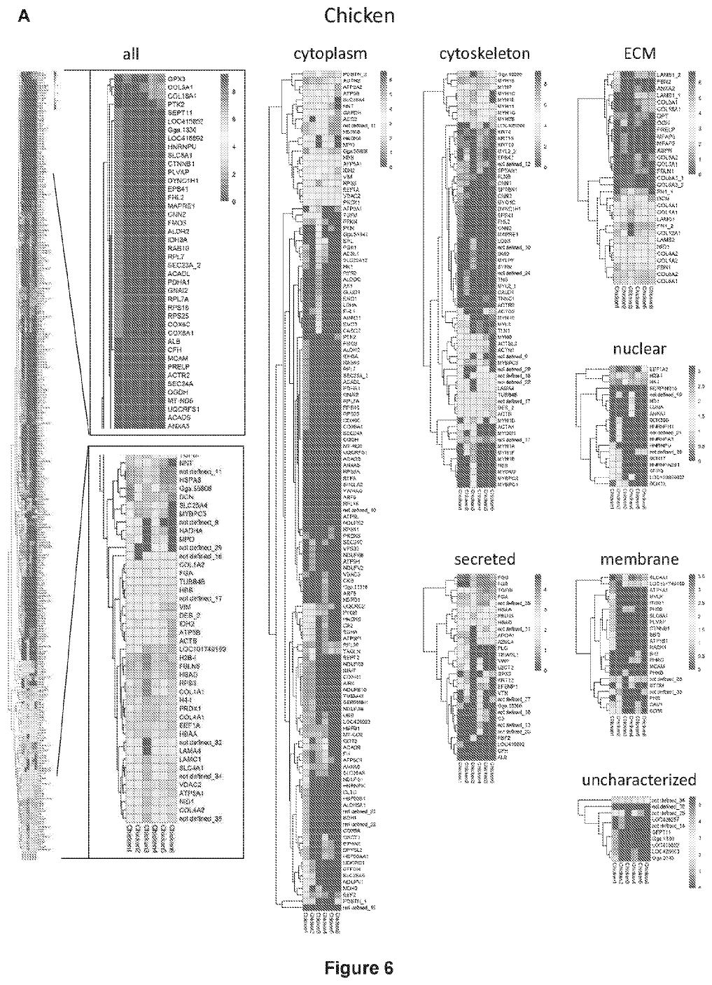

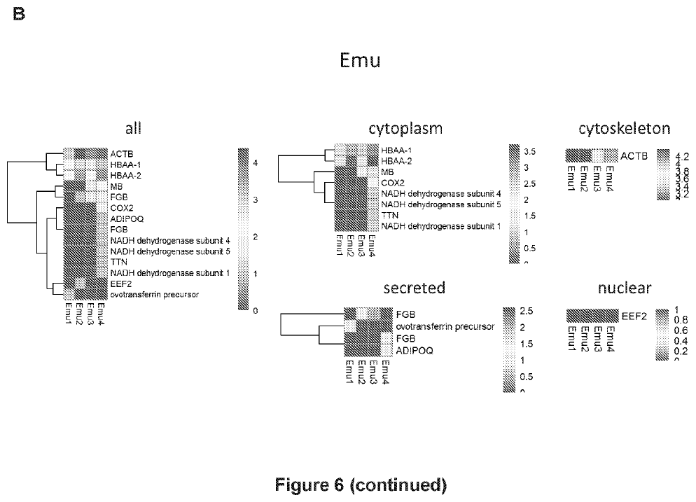

Proteins positively identified with two or more unique peptide hits by mass spectrometric analyses of decellularized chicken and emu lungs were subsequently categorized into one of six groups based on cellular or extracellular location: cytosolic, ECM, cytoskeletal, nuclear, membrane-associated, or secreted. In rare cases no classification could be assigned and therefore proteins were grouped as “uncharacterized”. Heatmaps generated from the log 2 transformation of unique peptide hits from each positively identified protein are depicted for visual comparison in FIG. 6. The number of total proteins identified in chicken and emu lungs is limited by available databases, particularly for emu lungs. As such, a total of 307 proteins were detected in decellularized chicken lungs with a variation of about 25% between individual lungs and detection of 185±46 proteins/lung on average. Only 14 proteins were identified in decellularized emu lungs with a variation of about 37% between individual emu lungs and detection of 9±3 proteins/lung on average. Individual peptide counts are depicted in FIGS. 14 and 15. Notably, no ECM proteins were identified in decellularized emu lungs, again reflecting limitations in available databases.

Human Lung Cells Survive for Varying Times Following Inoculation into Decellularized Chicken Vs Emu Lungs.

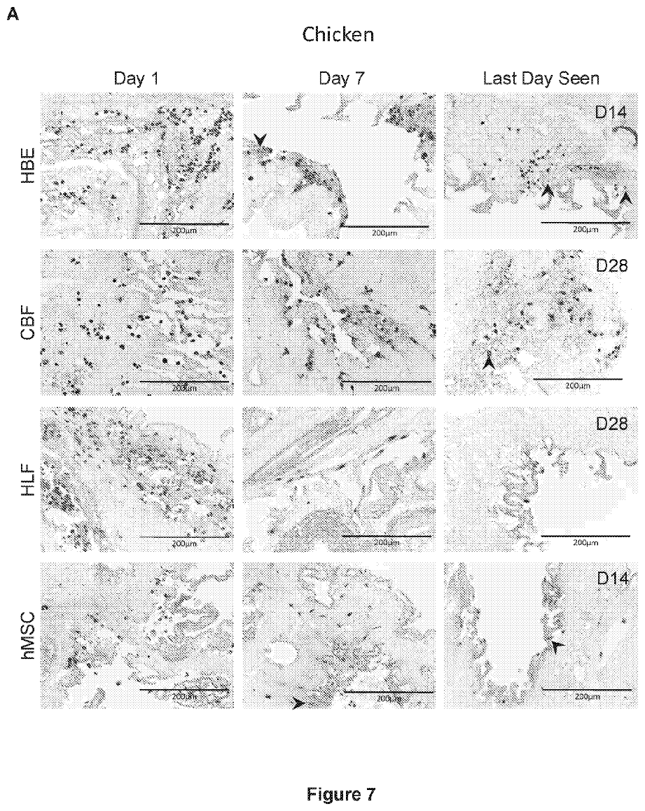

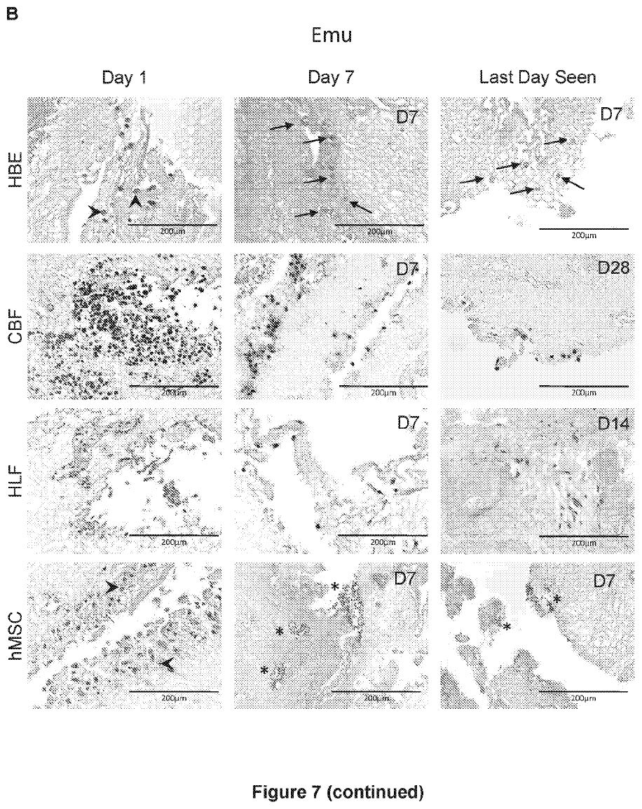

Using recellularization techniques previously described for both small and large mammalian models, inoculation of decellularized chicken and emu lungs was performed via a major airway or vascular conduit to either the entire organ (chicken) or 2-3 cm³ segments (emu). Initial cell binding, cellular localization and adhesion to the extracellular matrix, and cellular growth of the human cell lines were assessed histologically at each time point. Representative images of days 1, 7, and the last day on which viable-appearing cells were observed on the scaffolds, are displayed in FIG. 7. All cells initially adhered comparably to decellularized chicken scaffolds on day 1 and could be found through at least day 14 (day 28 for CBF and HLF). In contrast, initial seeding (day 1) of both HBEs and hMSCs demonstrated less robust appearance on the emu scaffolds and many cells appeared to be undergoing apoptotic changes. HBE cells were nearly absent of nuclear staining on day 7 on the emu lungs (FIG. 7B) and were only seen until day 7 compared to day 14 on the chicken lungs. In comparison to day 1, hMSC cells were rounded up and small on day 7. They further were only viable until day 7 on emu compared to day 14 on the chicken scaffolds. Thereby, the last day viable cells were seen on the decellularized emu lungs was generally shorter than on the chicken lungs. CBFs and HLFs demonstrated robust initial attachment to the emu scaffolds on day 1 and were viable until day 14 (HLF) and day 28 (CBF), respectively.

Assessment of Proliferation (Ki67-Staining) and Apoptosis (Caspase 3-Staining) of the Cells after Initial Attachment on Day 1 and after 7 Days of Incubation Show Cells Proliferating on Chicken Scaffolds and Apoptosing on Emu Tissue.

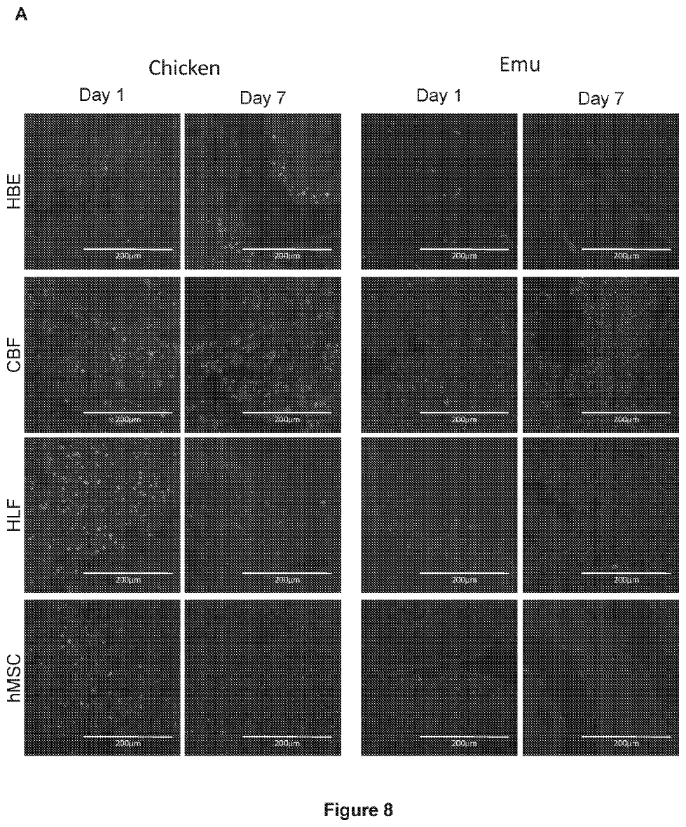

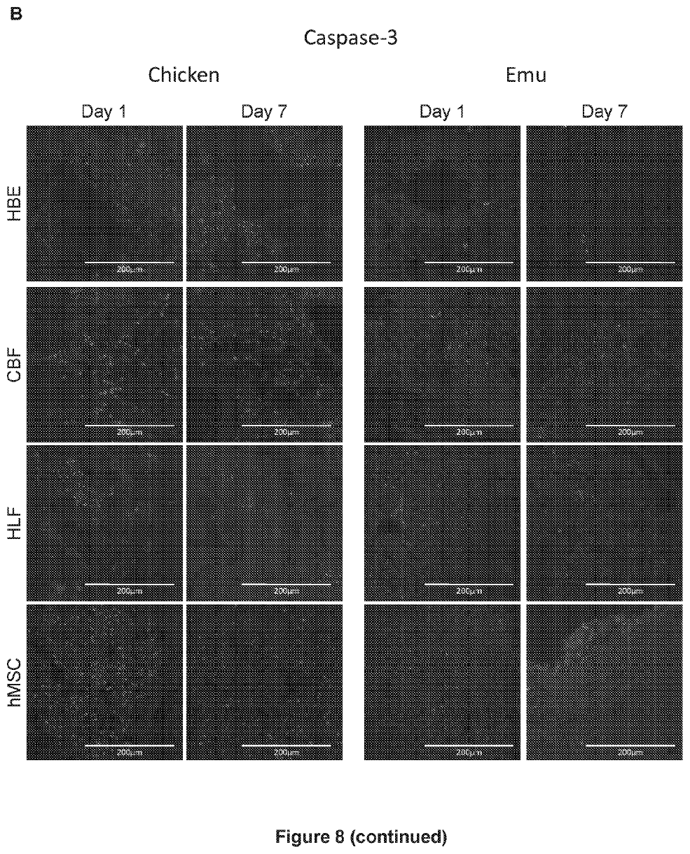

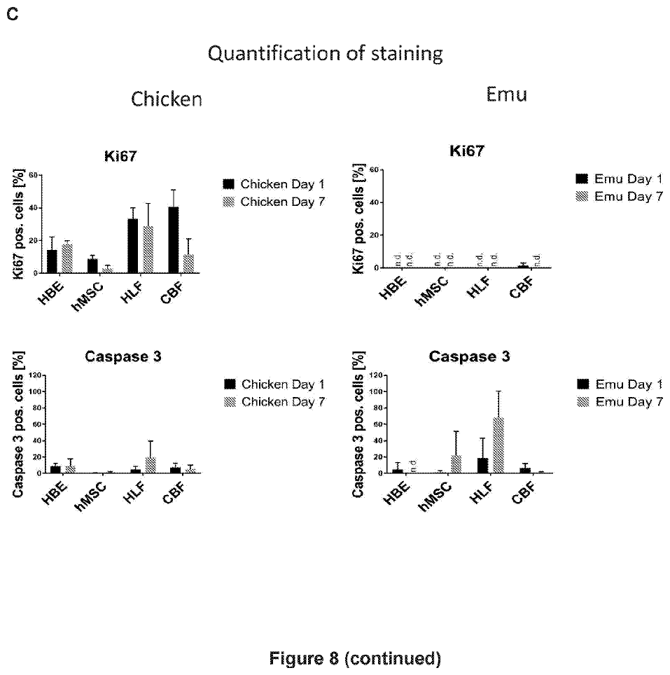

Initial Ki-67 staining (day 1) of the cells inoculated into the chicken scaffolds ranged between 10 and 40% with highest values for HLF and CBF cells (FIG. 8). At day 7 no significant difference compared to day 1 was seen for all cell types although there was a trend to lower Ki67 staining especially for the CBF cells. Caspase 3 staining was generally low for all cell types seeded onto the chicken scaffolds and was comparable between day 1 and day 7.

In contrast, little Ki67 staining was observed for any cell type seeded into the emu scaffolds on either day 1 or 7. However, significant amounts of caspase-3 staining was observed for all cell types on both day 1 and day 7. Further, there was a trend towards increased caspase-3 staining of HLFs and hMSCs on day 7.

### Example 2

This example provides another illustration of the decellularization and recellularization processes.

Decellularization

Chicken heart and lungs were harvested en bloc. The right and left lung were identified and separated from the heart in order to have isolated lobes. After separation the pulmonary artery (PA), pulmonary vein (PV) and main bronchi (B) (FIG. 16) were cannulated using a ⅛″ female Luer connector (Cole-Parmer Instrument). Each lung was flushed three times with each solution (250 mL per flush) using a continuous flow rate of 0.5 L/min alternately, via bronchi (airway) and pulmonary artery (vasculature). The following reagents were perfused sequentially using a roller pump (Stockert Shiley) under sterile conditions: 1× Phosphate Buffered Saline (PBS) (Corning, Corning, N.Y., USA) to clear mucus and blood, deionized water (DI), 0.1% Triton X-100 (Sigma-Aldrich, St. Louis, Mo., USA) solution and 24 h incubation at 4° C., 2% sodium deoxycholate (SDC, Sigma-Aldrich) solution and 24 h incubation at 4° C., 1M sodium chloride (NaCl, Sigma-Aldrich) solution and 1 h incubation at room temperature (RT), DNAse solution (30 mg/L bovine pancreatic deoxyribonuclease, 1.3 mM MgSO₄, 2 mM CaCl2 in DI water, all Sigma Aldrich) and 1 h incubation at RT, sterilization solution (0.1% peracetic acid (Sigma-Aldrich) in 4% ethanol) and 1 h incubation at RT and storage solution (1×PBS supplemented with Penicillin/Streptomycin (500 IU/mL Penicillin/500 μg/mL Streptomycin, Lonza, Basel, Switzerland), Gentamicin (50 mg/L, Corning), and Amphotericin B (2.5 mg/L, Corning)). (FIG. 17). DI rinses were performed between reagents and lungs were kept in agitation during incubation periods. The lung can be stored in PBS at 4° C. at least up to a month until recellularization.

Recellularization

Whole chicken lungs were connected to a custom-built bioreactor system which allows continuous media perfusion at different flow rates through the vasculature and airway. The system was placed in a standard incubator at 37° C. with 5% CO₂. Lungs were coupled to the system using the PA, PV and B connectors. Recellularization was performed in 3 individual lungs per cell type. Lungs were perfused with PBS and then cell culture medium at a continuous flow rate of 4 ml/min. The cells used were: Human bronchial epithelial (HBE), human lung fibroblasts (HLF), human pulmonary vascular endothelial (CBF, HUVEC), or human mesenchymal stromal (hMSC). Cells were suspended in 40 ml of the medium. HBE or HLF were seeded via the airway and CBF, HUVEC, or hMSC via the vasculature in a bioreactor cultivation system for up to 3 days. Overnight the flow rate was kept low at 2 mL/min through B, 1 mL/min trough PA and PV open to atmosphere for the seeding through airway, and at 1 mL/min through PA and PV, and B open to atmosphere for those cell infused via vasculature. This features allowed to perfuse cell cultivation medium at 37° C. overnight and cellular attachment. The following day flow rates were increased to 6 mL/min in B and 4 mL/min in PV, and 4 mL/min and PV open to atmosphere for airway and vasculature seeding, respectively. Cell cultivation media was replaced once after 4 h post-inoculation.

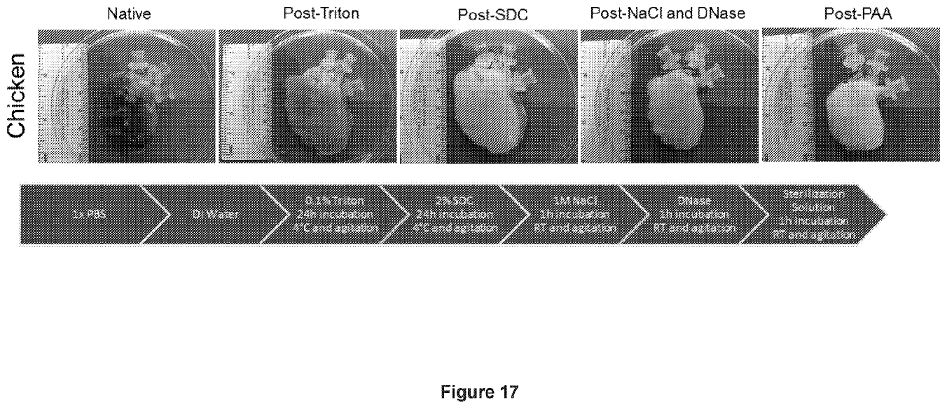

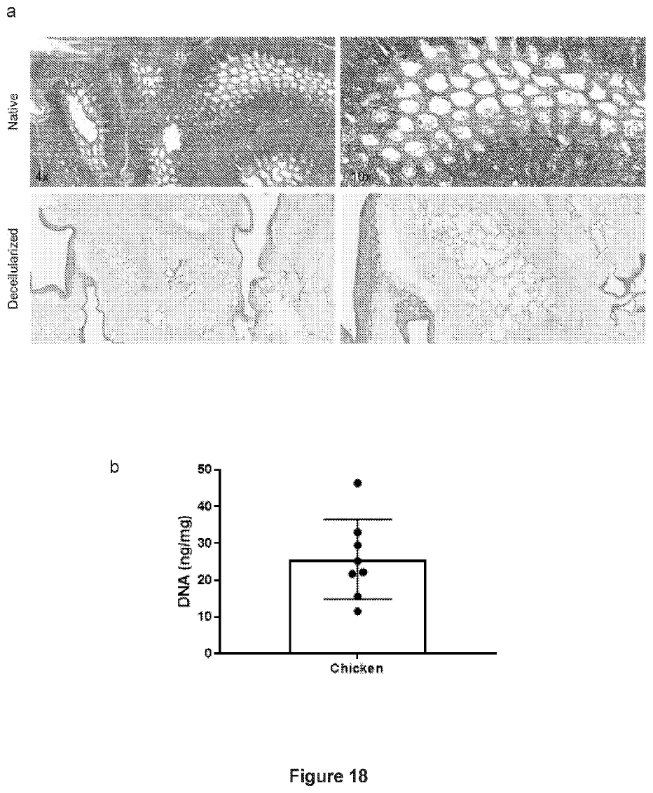

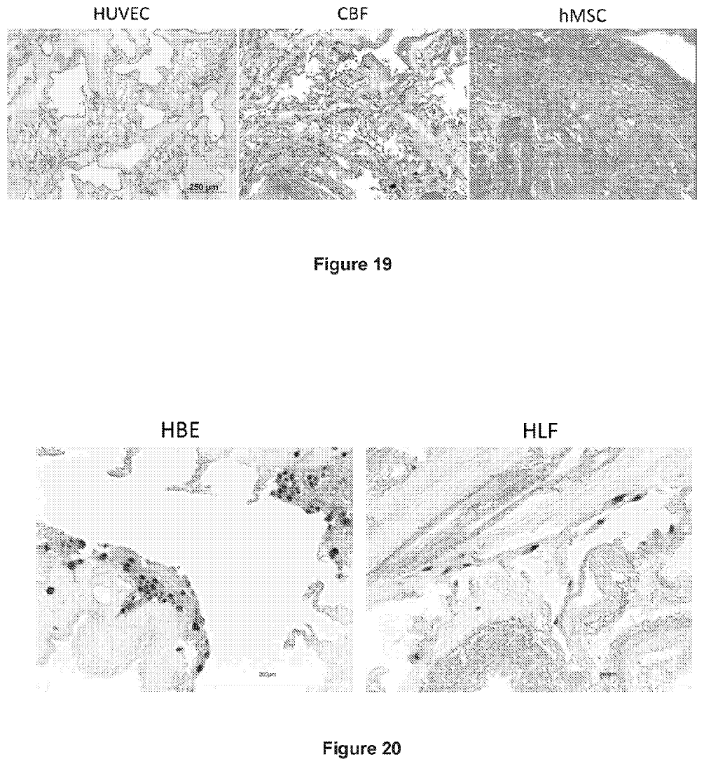

H&E staining (FIG. 18a) shows the exposed microarchitecture of the chicken lungs that is preserved after decellularization process. Parabronchi preservation (hexagonal-mesh-structure) can be clearly visualized at 4× (left) and 10× (right) magnification. Quantification of remaining DNA (FIG. 18b) in acellular chicken lungs certified the removal of DNA, reducing from 239.2±64.88 to 25.85±9.94 ng/mg after the decellularization process. Absence of cell nuclei were confirmed by H&E staining. Decellularized whole chicken lung scaffolds were cultured in a custom-built bioreactor system. These lungs were used to mimic the 3D environment for cells. After 3 days of culture, CBF and HUVEC cells demonstrated initial repopulation of the scaffold (FIG. 19). They were located in the vascular compartments and their morphology indicates cell growth. HMSC cells infused through the vasculature showed infiltration into the ECM, migrating from the vascular bed into the gas exchange tissue, with an augmented colonization of this region in the lung (FIG. 19). HBE cells perfused through the airway can be seen attached to the main parabronchi (FIG. 20). HLF cells perfused by the bronchi demonstrated attachment to the ECM with no migration into the gas exchange tissue. These findings collectively (FIG. 19 and FIG. 20) show that the acellular chicken lung scaffold preserves mostly biochemical, structural and mechanical cues which promotes cell attachment, growth, migration and repopulation.

While the present invention has been described through various specific embodiments, routine modification to these embodiments will be apparent to those skilled in the art, which modifications are intended to be included within the scope of this disclosure.

What is claimed is:

1. An isolated avian lung, which has been decellularized, and then recellularized with mammalian cells.

2. The avian lung of claim 1, wherein the mammalian cells comprise mammalian lung cells.

3. The avian lung of claim 2, wherein the mammalian lung cells are human cells.

4. The avian lung of claim 3, wherein the human cells are epithelial cells, endothelial cells, fibroblasts, and/or mesenchymal cells.

5. The avian lung of claim 2, wherein the mammalian lung cells are bronchial epithelial cells, pulmonary vascular endothelial cells, lung fibroblasts, bone marrow-derived mesenchymal stromal cells and/or endothelial colony forming cells.

6. A device for lung supplementation or replacement, the device comprising:
an isolated avian lung, which has been decellularized, and then recellularized with mammalian cells;
a housing containing the avian lung;
a blood supply conduit fluidically coupled to a vasculature of the avian lung and configured to be connected to a vessel of a patient;
a blood return conduit fluidically coupled to the vasculature of the avian lung and configured to be connected to an artery of the patient;
a breathing gas supply conduit pneumatically coupled to a gas intake of the avian lung and configured to be connected to a source of breathing gas; and
an exhaust conduit pneumatically coupled to a gas outlet of the avian lung for exhausting the breathing gas.

7. The device of claim 6, wherein the blood supply conduit is coupled to the pulmonary artery of the avian lung.

8. The device of claim 6, wherein the blood return conduit is coupled to the pulmonary vein of the avian lung.
